(19) United States
(12) Patent Application Publication
(10) Pub. No.: US 2025/0312102 A1
(43) Pub. Date: Oct. 9, 2025

(54) POSITIONAL AID DEVICES, METHODS AND SYSTEMS USEFUL FOR MEDICAL INTERVENTIONS

(71) Applicant: Muffin Incorporated, West Lafayette, IN (US)

(72) Inventors: Jesse Roll, West Lafayette, IN (US); Elyssia Wellington, Avon, IN (US); Nathan Ooms, DeMotte, IN (US); Eric Brandner, West Lafayette, IN (US); Orville Bramwell, Bloomington, IN (US)

(73) Assignee: Muffin Incorporated, West Lafayette, IN (US)

(21) Appl. No.: 19/172,964

(22) Filed: Apr. 8, 2025

Related U.S. Application Data
(60) Provisional application No. 63/631,435, filed on Apr. 8, 2024.
(60) Provisional application No. 63/765,116, filed on Feb. 28, 2025.

(51) Int. Cl. A61B 34/10; A61B 34/00; A61B 34/20; A61B 90/00

(52) U.S. Cl. CPC A61B 34/10; A61B 34/20; A61B 34/25; A61B 90/08; A61B 90/39; A61B 2034/107; A61B 2034/2065; A61B 2090/0807; A61B 2090/3937; A61B 2090/3954; A61B 2090/3966

(57) ABSTRACT

The present disclosure provides positional aid devices useful in imaging procedures such as magnetic resonance imaging (MRI) procedures. In certain embodiments the positional aid devices comprise a flexible layer structure having a first side and a second side opposite the first side; and the flexible layer structure defining a plurality of thru-passages demarked by material that is visible in images produced by the imaging procedure. Medical products such as kits and combination products including the positional aid devices, and methods of manufacture and use, are also described. MRI systems and related methods are also described.

This application claims the benefit of U.S. Provisional Patent Application No. 63/631,435, filed on Apr. 8, 2024, and U.S. Provisional Patent Application No. 63/765,116, filed on Feb. 28, 2025, which are hereby incorporated herein by reference in their entirety.

### BACKGROUND

The present disclosure relates generally to devices, systems and methods that can be used to aid in conducting medical procedures guided by an imaging modality, preferably magnetic resonance imaging (MRI), and in more particular aspects to such devices, systems and methods that can be associated with a region of a patient to assist in processing and assessing image data and/or facilitating actions taken during the medical procedure.

In performing medical procedures under MRI or other imaging modalities, ensuring efficiency and/or the efficacy is paramount for patient care. Locating a precise operating point on patient tissue in relation to an internal tissue region of interest, and providing inputs to a clinician or other health care worker to facilitate placement and/or orientation of procedural devices, advantageously increase the efficiency and/or the efficacy of the procedures. Current approaches to locating the operating point on a patient during an MRI guided procedure include laser indicating systems, whereby the intersection of a series of laser sheets are utilized to illuminate an operating point on the patient tissue; flexible sheets with markers, whereby a target operating site can be calculated and marked with ink between discrete markers; and/or the “finger poke” method, whereby a clinician deforms the patient tissue surrounding a proposed operating point to determine the actual operating point under MRI guidance. While these current approaches are able to locate an operating point on patient tissue, they provide limited or no MRI signals ex vivo. Therefore, these approaches require clinicians to manually position the medical devices on and/or above the patient tissue under limited or no MRI guidance.

Thus, there remain needs for improved and/or alternative devices, systems and methods to reinforce clinicians' efficiency and/or the efficacy in performing medical procedures under MRI or other imaging guidance. Aspects of the present disclosure are addressed to those needs.

### SUMMARY

In certain aspects herein, provided are magnetic resonance imaging (MRI) positional aid devices. The devices include a flexible layer structure having a first side and a second side opposite the first side. The flexible layer structure defines a plurality of thru-passages, wherein the thru-passages extend completely through a thickness of the flexible layer structure, and wherein the thru-passages are demarked by an MRI visible material of the flexible layer structure. The thru-passages can be in a defined array and/or can each form a concavity of decreasing dimension as it extends from the first side toward the second side. The flexible layer structure can have a thickness of at least 0.5 cm, for example in the range of about 0.5 cm to about 2 cm, in some aspects.

In other aspects herein, provided are positional aid devices for use with a medical imaging system. The devices include a layer structure having a first side and a second side opposite the first side, the layer structure defining a plurality of thru-passages that extend completely through a thickness of the layer structure. The thru-passages are demarked by a visible material of the layer structure visible in images generated by the medical imaging system. The thru-passages of the plurality of thru-passages each form a first concavity at the first side of decreasing dimension as it extends from the first side toward the second side. The layer structure can include one or more internal cavities and the material visible in images generated by the medical imaging system is contained in the one or more cavities. The layer structure can have a thickness of at least 0.5 cm, for example in the range of about 0.5 cm to about 2 cm, in some aspects.

In additional aspects herein, provided are procedural systems that include a positional aid device, and a guide device for guiding an interventional instrument. The positional aid device includes a layer structure having a first side and a second side opposite the first side, and defines a plurality of thru-passages that extend completely through a thickness of the layer structure. The thru-passages are demarked by a visible material of the layer structure visible in images generated by the medical imaging system. The guide device includes g a visible material visible in images generated by the medical imaging system. The positional aid device can be any of those as disclosed herein, including for example as described in this Summary above. The visible material of the guide device can demark an elongate longitudinal path along a length of the guide device.

In additional aspects herein, provided are MR imaging coil devices configured for use with a positional aid device. The MRI coil devices include a flexible base layer having a thru-opening, and a loop antenna positioned in the base layer, preferably around the thru-opening. The MR imaging coil devices also include at least one mounting frame attached to the base layer and defining a frame opening aligned with the thru-opening of the base layer and configured to receive a positional aid device. The MRI imaging coil device, and preferably the at least one mounting frame thereof, includes at least one fixation actuator movable between a first position configured to fix the positional aid device to the mounting frame while received in the frame opening and a second position configured to unfix the positional aid device from the mounting frame. The fixation actuator can include at least one projecting member configured for contacting the positional aid device when the fixation actuator is moved from the first position to the second position. In some forms, the at least one projecting member is configured for receipt within a fixation opening of the positional aid device when the fixation actuator is moved from the first position to the second position. The MRI imaging coil can also include the positional aid device, for example any of those disclosed herein.

In additional aspects herein, provided are methods, systems, kits and devices that incorporate or involve the use of positional aid devices, procedural systems, MRI coil devices, and other embodiments disclosed herein.

In still further aspects herein, provided are MR imaging methods, MRI systems, and devices for controlling MRI systems, that include steps or that are configured to automate or facilitate operations using a computer processor (e.g. of a computer), and that may involve positional aid devices, procedural systems, MRI coil devices and/or other embodiments disclosed herein.

Additional aspects or embodiments and features and advantages thereof will be apparent from the detailed description and drawings included herein.

### BRIEF DESCRIPTION OF THE DRAWINGS

FIG. 1 is a perspective view of a positional aid device comprising a flexible layer structure with a plurality of openings according to one embodiment.

FIG. 2 is a side cross-sectional view of the positional aid device of FIG. 1 taken along line A-A and viewed in the direction of the arrows.

FIG. 3 is a partial cutaway perspective view of the positional aid device of FIG. 1 taken along line A-A.

FIG. 4 is an enlarged cutaway top view of the upper right-most 3X3 opening grid of the positional aid device of FIG. 1 and illustrating internal passage paths in dotted lines.

FIG. 5 is an enlarged cutaway view showing a portion of the cross section of FIG. 2 including an illustrative opening of the positional aid device thereof.

FIG. 6 is a perspective view of an embodiment of an interventional device including a guide device and a tissue penetrating device usable with the positional aid device of FIG. 1.

FIG. 7 shows the cross sectional portion of FIG. 5 of the positional aid device received against skin of a patient and its illustrative interaction with a tip portion of the interventional device of FIG. 6.

FIG. 8 shows the view of FIG. 7 after adjustment of the orientation of the interventional device relative to the positional aid device.

FIG. 9 shows the view of FIG. 8 after advancement of the tissue penetrating device relative to the guide device to extend through an opening of the positional aid device and penetrate the skin of the patient.

FIG. 10 is a partial top view of another embodiment of a positional aid device.

FIG. 11 is a perspective view of another embodiment of a positional aid device.

FIG. 12 is a side elevational view of one of a plurality of cover inserts of the positional aid device of FIG. 11.

FIG. 13 is a side cross-sectional view of the positional aid device of FIG. 11 taken along line C-C and viewed in the direction of the arrows.

FIG. 14 is a partial cutaway perspective view of the positional aid device of FIG. 11 taken along line B-B.

FIG. 15 is a perspective view of a positional aid device incorporated with an MRI surface coil.

FIG. 16 is a perspective view of the MRI surface coil of FIG. 15 adapted for combination with the positional aid device.

FIG. 17 is a partial cutaway perspective view of the MRI surface coil of FIG. 16 illustrating internal features.

FIG. 17A is a cross-sectional view taken along line 17A-17A of FIG. 16 and viewed in the direction of the arrows.

FIG. 18 is a perspective view of the positional aid device of FIG. 15.

FIG. 18A is a cross-sectional view taken along line 18A-18A of FIG. 18 and viewed in the direction of the arrows.

FIG. 19 is a perspective view of the positional aid device and MRI surface coil of FIG. 15 prior to movement of a fixation actuator to a position that fixes the positional aid device to the MRI surface coil.

FIG. 20 is a schematic illustration of an MRI system in accordance with embodiments herein.

FIG. 21 shows a digital image providing an example two-dimensional MRI slice image showing a positional aid device and a region of interest in a non-clinical model, with annotations.

FIGS. 22A-H show digital images providing exemplary two-dimensional MRI images at sequential procedural stages and including a positional aid device, an MRI-visible interventional apparatus used with the positional aid device, and a region of interest in a non-clinical model, with annotations.

FIG. 23A-D show digital images providing exemplary MRI images of a maximum intensity projection (MIP) image (FIG. 23A) and several parallel two-dimensional MRI image slices (FIGS. 23B-D) used to generate the MIP image.

FIG. 24 shows a digital image providing an example two-dimensional MRI slice image showing a positional aid device and a region of interest in a non-clinical model, with annotations.

FIG. 25A provides a simplified schematic illustrating workflow for one embodiment of an MRI-guided procedure of the present disclosure.

FIG. 25B provides a simplified schematic illustrating workflow for another embodiment of an MRI-guided procedure of the present disclosure, optionally supplemental to that of FIG. 25A.

FIG. 26 provides a perspective view of one embodiment of a magnetic resonance imaging positional aid device as disclosed herein.

FIG. 27 provides a perspective view of one embodiment of a magnetic resonance imaging positional aid device as disclosed herein.

FIG. 28 provides a perspective view of one embodiment of a magnetic resonance imaging positional aid device as disclosed herein.

FIG. 29a provides a cross-sectional side view of a portion of one embodiment of a magnetic resonance imaging positional aid device as disclosed herein.

FIG. 29b provides a cross-sectional side view of a portion of one embodiment of a magnetic resonance imaging positional aid device as disclosed herein.

FIG. 30 provides a perspective view of one embodiment of a magnetic resonance imaging positional aid device as disclosed herein.

FIG. 31 provides a perspective view of one embodiment of a magnetic resonance imaging positional aid device as disclosed herein.

### DETAILED DESCRIPTION OF SELECTED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications in the described embodiments and any further applications of the principles of the disclosure as described herein are contemplated as would normally occur to one skilled in the art to which the disclosure relates. A number of embodiments of the disclosure are shown in great detail, although it will be apparent to those skilled in the relevant art that some features that are not relevant to the present disclosure may not be shown for the sake of clarity.

As disclosed above, aspects of the present disclosure relate to a positional aid device useful for facilitating magnetic resonance imaging (MRI) procedures. For example, positional aid devices disclosed herein may be useful for MRI-guided placement of a needle shaft or other tissue penetrating instrument into a patient during a medical procedure such as a therapeutic and/or diagnostic medical procedure. In this regard, while certain disclosures below and elsewhere herein discuss the placement of a needle shaft, it will be understood that such disclosures can also apply to the placement of other tissue penetrating instruments configured for penetration into and through patient tissue. The needle shaft or other tissue penetrating instrument can, for example, be configured for use in an in-bore procedure, such as a biopsy of patient tissue, and/or for administering a diagnostic and/or therapeutic substance to patient tissue, which may, for example, be a liquid substance, through a lumen thereof. Additionally, while discussions herein focus on guided procedures conducted under MRI, it will be understood that in certain aspects embodiments herein can be useful for and/or used in guided procedures conducted under other imaging modalities, for example X-ray guided procedures such as fluoroscopically or computerized tomography (CT) guided procedures. As examples, a material of a positional aid device visible under MRI as discussed herein may also be visible under, or may be replaced or supplemented with a material visible under, another imaging modality, for example a radiopaque material in the case of X-ray guided procedures. These and other aspects will be apparent to those skilled in the field from the descriptions herein.

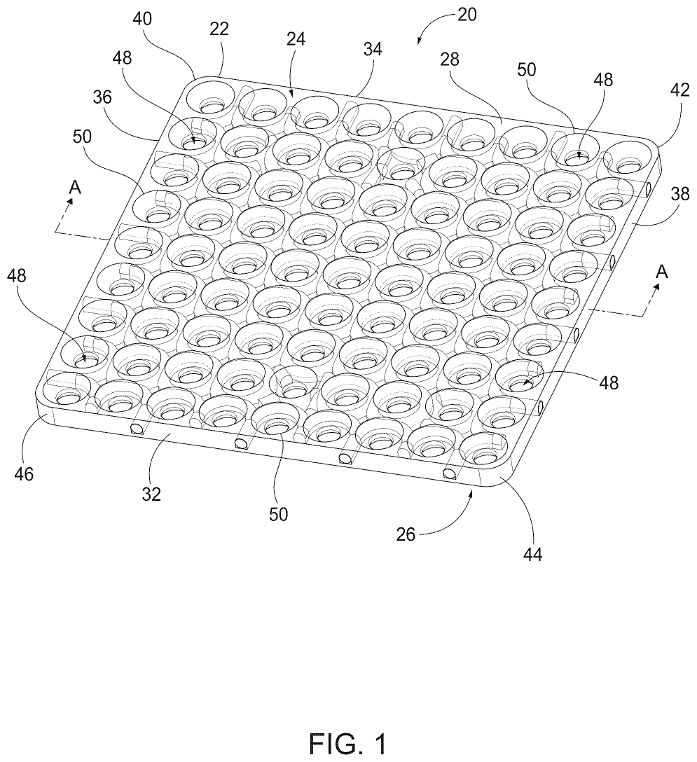

Turning now to a discussion of the figures, FIG. 1 provides a perspective view of a positional aid device 20 according to one embodiment herein. Positional aid device 20 is at least partially visible under MRI. As shown, the positional aid device 20 includes a flexible layer structure 22. The flexible layer structure 22 can comprise a flexible material incorporated such that the positional aid device 22 can conform to a curvilinear surface such as a patient tissue (e.g. skin) surface, for example in some forms under the force of gravity. The flexible layer structure 22 can incorporate an MRI visible material in any suitable way. These include, as examples, an MRI visible material contained in one or more internal voids defined by the layer structure, and/or an MRI visible material distributed (e.g. substantially homogenously distributed) within a solid matrix material forming all or a portion of the layer structure 22, and/or a coating(s) applied to a solid matrix material of the layer structure 22.

As shown, the flexible layer structure 22 of the illustrated embodiment includes a first side 24 and a second side 26 opposite the first side 24. The first side 24 typically faces away from tissue of the patient and toward a user of the positional aid device 20. The first side 24 defines a first surface 28. As discussed further herein, the first surface 28 can have features configured to facilitate medical procedures, such as in-bore percutaneous interventions. The second side 26 is configured to face toward patient tissue, such as skin. The second side 26 defines a second surface 30. In one version, the second surface 30 includes an adhesive surface or layer (not shown) on all or a portion thereof configured to temporarily maintain the positional aid device 20 at a fixed position on patient tissue. The adhesive surface or layer may include any suitable configuration or adhesive. The adhesive may for example be a pressure-sensitive adhesive, and in some forms may be a dry adhesive configuration to the surface that utilizes Van der Waal interactions that promote surface adhesion.

The flexible layer structure 22 further defines a first edge 32, a second edge 34 opposite the first edge 32, a third edge 36, and a fourth edge 38 opposite the third edge 36. As will be appreciated, in the illustrated embodiment each of the first edge 32, the second edge 34, the third edge 36, and the fourth edge 38 extends between and connects the first surface 28 and the second surface 30. According to one example, the flexible layer structure 22 has a substantially constant thickness and the first edge 32, the second edge 34, the third edge 36, and the fourth edge 38 all have a same height dimension. In some forms, the flexible layer structure 22 has a thickness of less than about 2 centimeters, or less than about 1 centimeter; in addition or alternatively, the flexible layer structure 22 can have a thickness of at least about 0.2 centimeters, or at least about 0.5 centimeters. Embodiments wherein the flexible layer structure 22 has a thickness of at least about 0.5 centimeters will be particularly beneficial in configurations constructed for interaction with other medical instruments, e.g. as described herein, as they can provide significant surfaces extending transverse to the first surface 28 and/or the second surface 30 for abutment against and interaction with the instruments (e.g. to stabilize a position and/or limit a range of motion thereof relative to the positional aid device 20).

The flexible layer structure 22 can have any suitable size or shape when considered in a plane parallel to surface 28 and/or 30. Typically, the flexible layer structure will have a length (e.g. along edges 32 and 34) and/or a width (e.g. along edges 36 and 38) in the range of about 5 cm to about 20 cm and/or will be sized to cover a surface area in the range of about 25 cm² to about 400 cm². The flexible layer structure 22 may define a generally polygonal periphery (e.g. rectangular, including as an example square), a generally circular periphery, a generally ovoid periphery, or an irregular periphery. As illustrated, where the flexible layer structure defines a generally polygonal periphery such as a rectangle, it can include radiused corners 40, 42, 44 and 46 joining adjacent edges of the periphery.

It will be understood that the flexible layer structure 22 may have other thickness, size, or shape configurations than those described above. For example, the layer structure may have a varying thickness and/or may have a thickness, length, width or surface area coverage that is less than or greater than those described above.

In the illustrated embodiment, the flexible layer structure 22 of the positional aid device 20 defines a plurality of thru-passages 48 arranged in an array, for example in some embodiments defining from about four to about two hundred and twenty five thru-passages 48. Thru-passages 48 extend completely through the thickness of the layer structure 48. Each of the thru-passages 48 includes a first rim 50 located at the first surface 28 and a second rim 52 located at the second surface 30. In the illustrated embodiment, each of the thru-passages 48 has a generally circular cross-sectional shape in a plane perpendicular to the axis of the thru-passage 48, and such a circular cross-sectional shape or other cross-sectional shapes that define a continuously curved circumference (e.g. an ovoid cross-sectional shape) are considered as preferred. It will be understood, however, that in other embodiments the thru-passages can have another cross-sectional shape in a plane perpendicular to the axis of the thru-passage, for example a polygonal cross-sectional shape, and/or that the layer structure 22 can define thru-passages of multiple, differing cross-sectional shapes as disclosed herein.

In some forms, the array of thru-passages 48 includes thru-passages 48 arranged in a two-dimensional grid, where the grid includes a first set of thru-passages 48 providing multiple (i.e. two or more) rows of thru-passages 48 extending parallel to a first axis (e.g. an axis “X”) and a second set of thru-passages 48 providing multiple columns of thru-passages 48 extending parallel to a second axis transverse to, and preferably perpendicular to, the first axis (e.g. an axis “Y”). The illustrated positional aid device 20 provides an example of a layer structure 22 including eighty one thru-passages 48 in a 9×9 (i.e. “nine by nine”) grid array, with nine rows of nine thru-passages extending parallel to a first axis and nine rows of nine thru-passages 48 extending parallel to a second axis perpendicular to the first axis. In other embodiments, the layer structure 22 may define a similar 2×2, 3×3, 4×4, 5×5, 6×6, 7×7, 8×8, 10×10, 11×11, 12×12, 13×13, 14×15, or 15×15 grid, as examples. It will be understood that the number of thru-passages in each row may be the same or different from the number of thru-passages in each column in a grid array. Grid arrays in which each row includes at least three thru-passages 48 and in which column includes at least three thru-passages 48 are preferred. It will also be understood that the layer structure 22 may define thru-passages additional to those thru-passages of a particular defined grid or other array of the layer structure 22. In some forms, the array of thru-passages is configured to facilitate a wide range of interventional procedures. Uniform two-dimensional grid arrays as discussed above may be beneficial for these purposes. In other forms, the array of thru-passages 48 may be configured to facilitate a specific procedure or type of procedure. For example, the thru-passages 48 may be arrayed with positions and spacing from one another to facilitate the use of two or more interventional instruments each passed through a respective thru-passage 48. Illustratively, in procedures wherein it is desired that the distal regions or other features of two separately introduced instruments are spaced and/or oriented in a particular manner relative to one another within patient tissue, the thru-passages 48 may be positioned on the device 20 and/or may have axes configured to help direct those distal regions or other features to the desired spacing and/or orientation. As one example, some known tissue ablation procedures employ two (or more) separately introduced ablation needles wherein certain positioning of respective distal regions of the needles relative to one another is desired.

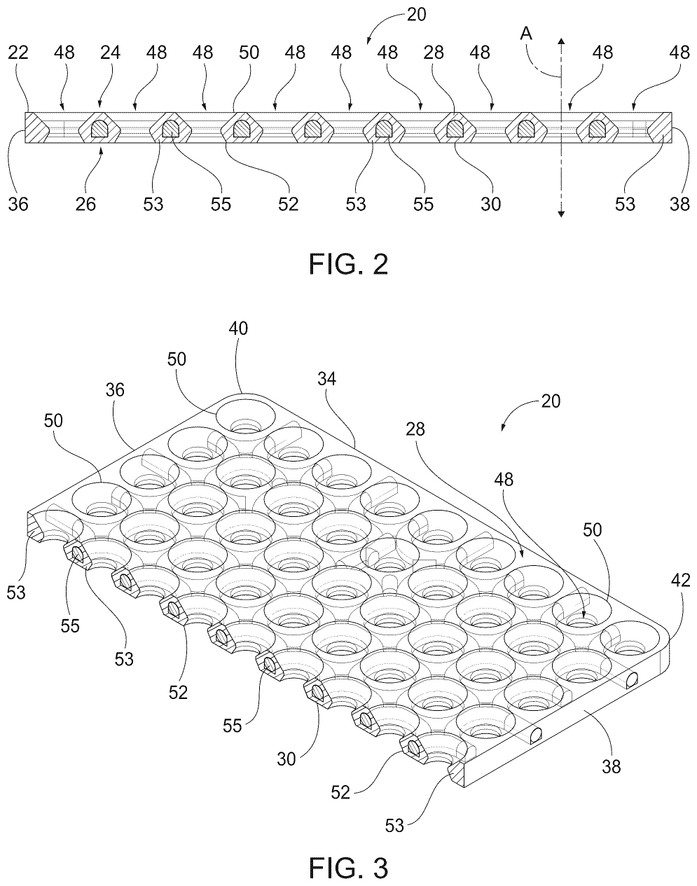

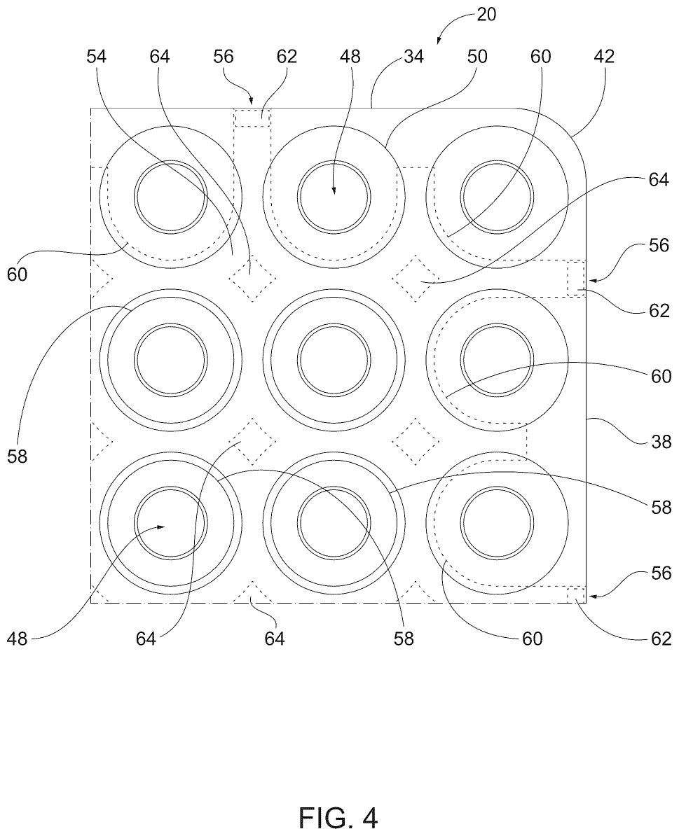

With continuing reference to FIG. 1 along with FIG. 2, FIG. 3 and FIG. 4, in the illustrated embodiment, the thru-passages 48 are demarked by MRI visible material of the flexible layer structure 22 so that the position of the thru-passages 48 can be visually determined on an MRI image of the positional aid device 20. Typically, the MRI visible material will include water, and in some forms will include water and at least one gel-forming agent. In the illustrated form, the flexible layer structure 22 includes a polymer matrix 53 that defines an internal passageway system 54 for containing the MRI visible material 55. A flexible layer structure 22 that defines such an internal passageway system 54 or another cavity or plurality of discrete cavities for containing the MRI visible material will be of particular advantage where the MRI visible material is, at least during its introduction into the flexible layer structure 22, in a form other than a three-dimensionally stable solid. For example, for introduction into the flexible layer structure 22, the MRI visible material can be in a flowable form such as an aqueous liquid, aqueous gel or aqueous semi-solid, which after introduction into the layer structure 22 (e.g. internal passageway system 54) can in some embodiments self-transition or be caused to transition to a three-dimensionally stable solid, for example by gelling, curing and/or the introduction of ionic or covalent crosslinks. In certain preferred forms, the MRI visible material is a water-containing material, particularly where the water-containing material also includes (i) a sodium salt, such as sodium chloride, and a polymer, such as polyacrylic acid, or (ii) vitamin E oil, or (iii) a gel-forming agent, such as gelatin or agarose, and at least one metal salt, such as copper sulfate, nickel sulfate, and/or thulium nitrate. In the illustrated embodiment, the internal passageway system 54 defines a plurality of connected internal passages configured to demark the thru-passages 48 with MRI visible material 55, and having ports 56 positioned on the edges 32, 34, 36 and 38 of the layer structure 22 providing communication with the internal passageway system 54.

FIG. 4 provides an enlarged top view of an upper right-hand region of the device 20 as illustrated in FIG. 1 showing the internal passageway system in dotted lines, to provide greater clarity. As shown therein, the internal passageway system 54 and thus the MRI visible material contained therein defines boundaries 58 that completely circumscribe some of the thru-passages 48 (all except those positioned along the edges of layer structure 22), and boundaries 60 that only partially circumscribe some of the thru-passages 48 (those positioned along the edges of layer structure 22). In the illustrated form, the boundaries 58 define a path that corresponds in shape (circular) to the outer circumference of the cross-sectional shape of the thru-passages (circular), and the boundaries 60 define a path that corresponds in shape (arc of a circle) to a portion of the outer circumference of the cross-sectional shape of the thru-passages (circular). It will be understood that layer structure embodiments 22 herein can include the illustrated or another passageway system that includes combinations of completely-circumscribing and partially-circumscribing MRI visible material boundaries around the thru-passages 48, all completely-circumscribing MRI visible material boundaries around the thru-passages 48, or all partially-circumscribing MRI visible material boundaries around the thru-passages 48, to demark the position of the thru-passages 48. In the case of completely-circumscribing MRI visible material 55 boundaries, the closed circular or other shape of the boundary of the MRI visible material can be positioned such that its center corresponds to a point along the axis “A” (see FIG. 2) of the corresponding thru-passage 48. In the case of partially-circumscribing MRI visible material boundaries, the path of the boundary or multiple discrete boundaries around the thru-passage can be extrapolated to a closed shape (e.g. a circle) the center of which corresponds to a point along the axis “A” of the corresponding thru-passage 48. While such closed shapes shown by or discernable from the MRI visible material 55 are beneficially corresponding in shape to the thru-passages, this is not necessary to all forms herein; for example, where thru-passages 48 are circular in cross-sectional shape, square shapes shown by or discernable from the MRI visible material may be positioned such that the center of the square shape corresponds to the center of the thru-passages 48, and vice versa. These and other variations by which the position of the thru-passages 48 may be demarked by the MRI-visible material will be apparent to those skilled in the field from the descriptions herein.

As shown most clearly in FIG. 4, the layer structure 22 can include a plurality of caps 62 that seal the ports 56, thus rendering the internal passageway system 54 fluid tight. As shown, the caps 62 can in some forms be positioned in the passageway system 54 proximate to ports 56. The use of caps 62 will be particularly beneficial in embodiments wherein the MRI visible material 55 is in a form other than a three-dimensionally stable solid such as a liquid, gel or semi-solid, as discussed herein, and the caps 62 can prevent leakage of the MRI visible material 55 from the internal passageway system 54. As also shown more clearly in FIG. 4, the internal passageway system 54 can be configured to leave a plurality of ribs 64 of the polymer matrix 53, positioned between the thru-passages and spanning from the first surface 28 to the second surface of the layer structure 22. The ribs 64 can for example be in the form of posts surrounded on all sides by the passageway system 54 and the MRI visible material contained therein. The ribs 64 can contribute to the structural integrity and flexural properties of the layer structure 22.

In one mode of making the positional aid device 20 of FIGS. 1-4, a flexible polymeric substrate defining the internal passageway system 54 can be manufactured as a single monolithic part or as multiple attached parts (e.g. layers of a laminate). Such manufacturing may involve, casting, molding, forming and/or other techniques. A flowable MRI visible material can then be introduced into the passageway system 54. For example, in some forms, caps 56 can first be incorporated and then the flowable MRI visible material can be injected into the passageway system through a cannulated device (e.g. a needle) temporarily inserted through the polymeric substrate at one or multiple locations (e.g. on surface 28 and/or 30). In other forms, a flowable MRI visible material can be introduced into the passageway system through one or more of the ports 56 while one or more of the ports 56 serve as a vent(s). This may occur with all vents 56 uncapped by caps 62, or where only some of the caps 62 have been installed. After filling with the MRI visible material, caps 62 can be installed as necessary to render the passageway system 54 fluid tight. These and other modes of manufacture of layer structures 22 herein will be apparent to skilled persons.

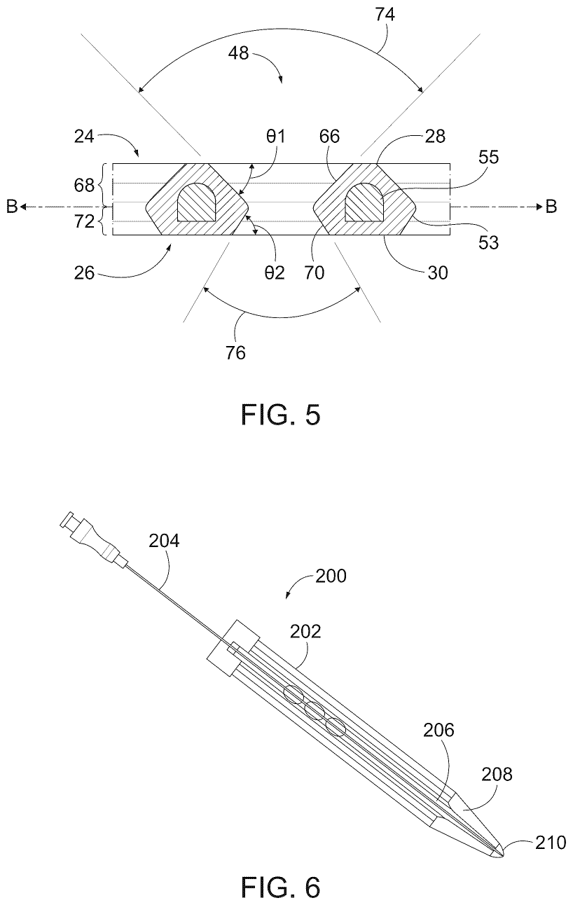

In preferred forms, the thru-passages 48 of the layer structure 22 will define concavities of decreasing dimension facing the first side 24 and/or the second side 26. In some embodiments, the concavities will be bowl-shaped or cone-shaped concavities and/or will each define a continuously curved cross-sectional shape in a plane perpendicular to the axis of the respective thru-passage 48 that defines the concavity, for example wherein such cross-sectional shape is a circular cross-sectional shape. With reference now to FIG. 5, provided is an enlarged cutaway view showing a portion of the cross section of FIG. 2 including an illustrative thru-passage 48 of the positional aid device 20. As can be seen, thru-passage 48 has a non-constant diameter as it extends through the thickness of layer structure 22. Thru-passage 48 defines a first concavity of decreasing dimension as it extends from the first side 24 toward the second side 26, and a second concavity of decreasing dimension as it extends from the second side 26 to the first side 24. Thru-passage has walls 66 that face toward first side 24 and extend transversely to first surface 28 and inwardly toward the central axis of the thru-passage 48. In particular, walls 66 extend at an angle θ1 relative to surface 28, where θ1 is less than 90 degrees and will typically range from about 20 to about 70 degrees, more preferably about 30 to about 55 degrees. Walls 66 can extend through the thickness of the layer structure 22 a distance 68 that represents only a portion of the thickness of the layer structure 22. In some forms, distance 68 represents at least about 10% of the thickness of the layer structure 22, and typically represents in the range of about 25% to about 75% of the thickness of the layer structure 22. Additionally or alternatively, distance 68 can be at least about 0.05 cm, or can be in the range of about 0.05 cm to about 1.5 cm. Thru-passage 48 also has walls 70 that face toward second side 26 and extend transversely to second surface 30 and inwardly toward the central axis of the thru-passage 48. In particular, walls 70 extend at an angle θ2 relative to surface 30, where θ2 is less than 90 degrees and will typically range from about 20 to about 70 degrees, more preferably about 45 to about 70 degrees. Walls 70 can extend through the thickness of the layer structure 22 a distance 72 that represents only a portion of the thickness of the layer structure 22. In some forms, distance 72 represents at least about 10% of the thickness of the layer structure 22, and typically represents in the range of about 25% to about 75% of the thickness of the layer structure 22. Additionally or alternatively, distance 68 can be at least about 0.05 cm, or can be in the range of about 0.05 to about 1.5 cm. In the illustrated embodiment, the thru-passage 48 has a smallest diameter and/or cross-sectional dimension that occurs between surface 28 and surface 30, an in particular between the first and second concavities discussed above, and in the specific illustrated embodiment within a plane that is parallel to surfaces 28 and 30 that incorporates line B-B (FIG. 5).

In some forms, the sum of distance 68 and distance 72 can equal the thickness of the layer structure 22. In other forms, the sum of distance 69 and distance 72 together can be less than the thickness of the layer structure 22, for example where thru-passages 48 may include a constant diameter segment intermediate to walls 66 and 70 and that has walls that are perpendicular to surfaces 28 and 30. These and other variations are possible. As will be discussed further below, the wall surfaces 66 can define a facilitated range of motion 74 for an instrument received thereagainst and wall surfaces 70 can define a facilitated range of insertion for an interventional tool to be introduced into a patient through thru-passage 48.

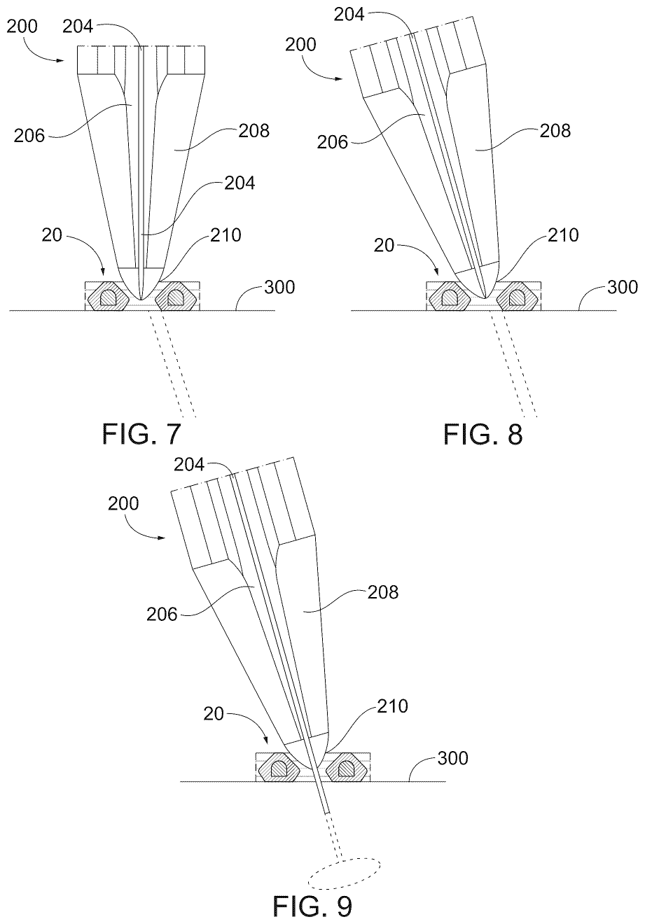

Referring now to FIG. 6, FIG. 7, FIG. 8 and FIG. 9, illustrated is one embodiment of an interventional device 200 that can be used in combination with positional aid device 20 and an exemplary mode of use thereof. Shown in FIG. 6 is interventional device 200 including a guide device 202 and an interventional instrument such as a tissue penetrating device, and in particular a needle 204, associated with the guide device 202. Guide device 202 can be a guide device, and needle 204 can be a needle, as described in the Applicant's co-pending U.S. Patent Application Ser. No. 63/588,561 filed Oct. 6, 2023 and entitled Device for MRI Guided Needle Placement, which is appended hereto as Appendix A and hereby incorporated herein by reference in its entirety. The illustrated guide device 202 defines a longitudinal groove 206 along a lateral side thereof configured to receive and guide an advancement path of a needle shaft or cannula of the needle 204, e.g. for insertion, and has an internal chamber that contains an MRI visible material that demarks the groove 206 in an image of the guide device 202 obtained by MRI (e.g. in an image plane taken parallel to and/or perpendicular to the longitudinal axis of the guide device), for example by providing a first amount of the MRI visible material positioned laterally to the groove to a first side and a second amount of the MRI visible material positioned laterally to the groove to a second side opposite the first side. Guide device 202 also defines a distal tip 210. In certain embodiments herein, the distal tip 210 and the walls 66 of the thru-passages 48 define respective surfaces configured to stabilize positioning and movement of the guide device 202 relative to the positional aid device 20. For example, the distal tip 210 can define a surface contoured to contact and conform to a surface defined by walls 66 of the thru-passages 48, preferably wherein such contact and conformance is maintained through a range of relative angular movement between the interventional device 200 and the positional aid device 20. In the illustrated form, the distal dip 210 defines a convexly rounded surface for contacting and conforming to concave wall surfaces of concavities respectively formed by thru-passages 48.

Referring now more particularly to FIGS. 7-9, shown are stages of an exemplary operation that can be carried out with the interventional device 200 and the positional aid device 20 placed against and potentially adhered to a surface 300, for example a skin surface of a patient. Shown in FIG. 7 is the interventional device 200 in an orientation generally perpendicular to the first surface 28 of the positional aid device 20, with the tip of the needle 204 positioned within the groove 206 of the guide device 202 and the convexly rounded distal tip 210 of the guide device 202 contacting and conforming with the walls 66 of one of the passages 48 of the positional aid device 20. In the illustrated embodiment, the size and cooperating surface configurations of the distal tip 210 and the passage walls 66 provide an interference stop to the distal tip 210 such that the distal tip 210 can penetrate only partially through the thickness of the positional aid device 20 and does not contact the surface 300. It will be understood that this is not necessary in all embodiments herein, and that in other forms the distal tip 210 and passage 48 may be sized and configured such that the distal tip 210 penetrates through the thru-passage 48 and contacts the surface 300.

Shown in FIG. 8 is a view of the arrangement shown in FIG. 7 after angular adjustment of the interventional device 200 relative to the positional aid device 20 while maintaining contact of the distal tip 210 with the passage walls 66 of a thru-passage 48 of the positional aid device 20. During such adjustment, an angle of a longitudinal axis of the interventional device 200 relative to the longitudinal axis of the thru-passage defining passage walls 66 contacted by the distal tip 210 can be altered, e.g. while such thru-passage remains in a stationary position relative to the patient. For example, the angular adjustment may be made to orient the groove 206 and needle 204 along a desired entry and travel path for the needle 204 below the surface 300, for example a path extending into patient tissue, and may involve angular adjustment in two dimensions. The desired entry and travel path may in some embodiments be provided in an MRI image by an MRI apparatus, and the angular adjustment may be made by a user with reference to MRI image(s) displayed in real time, as is discussed in more detail below. FIG. 9 shows the arrangement of FIG. 8 after distally-directed advancement of the needle 204 so that its distal shaft portion penetrates through surface 300, for example extending into patient tissue where surface 300 is a skin surface of a patient.

The use of the positional aid device 20 in combination with the interventional device 200, for example during the staged operations illustrated in FIGS. 7-9, and potentially other operations as discussed below, can be a part of a diagnostic and/or therapeutic procedure on a patient. Illustratively, a diagnostic procedure may be a biopsy procedure in which a patient tissue specimen is obtained (e.g. by aspiration or core sampling), and a therapeutic procedure may be a procedure in which an agent is delivered to patent tissue by the needle 204 or another tissue penetrating instrument. The agent may for example be an injectable drug or other therapeutic fluid delivered to patient tissue or energy (e.g. positive or negative thermal energy) delivered to patient tissue, for example as a tissue ablative agent.

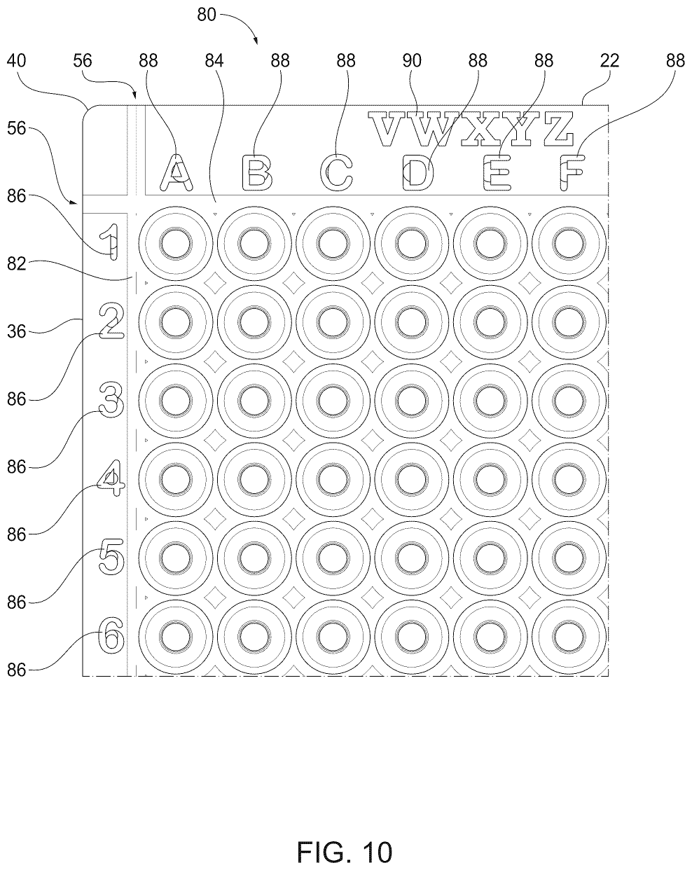

FIG. 10 illustrates a partial top view of the positional aid device 80 having features similar to device 20 discussed above. However, the flexible layer structure 22 of device 80 further comprises an MRI visible material-filled first internal passageway portion 82 extending along and demarking a first axis of the grid array of the thru-passage 48, and a second MRI visible material-filled internal passageway portion 84 extending along and demarking a second axis of the grid array of the thru-passages 48 perpendicular to the first axis. As well, device 80 includes a first series of indicia 86 extending parallel to the first axis and a second series of indicia 88 extending parallel to the second axis. The indicia 86 may in some forms be alphanumerical symbols. The indicia 86 and 88 may be MRI visible, for example provided by MRI visible material contained in correspondingly shaped cavities within the layer structure 22, and/or may be visible to the naked eye of a user (i.e. “directly visible”), for example ink stamped on or embossed in or on the surface of the layer structure 22. The indicia 86 and 88 can be configured to aid in the identification of a particular thru-passage 48 of the plurality of thru-passages 48. As shown, the indicia 86 include a plurality of differing numerical indicia in a defined series each marking a respective row of thru-passages 48 in the grid array, and the indicia 88 include a plurality of differing letter indicia in a defined series each marking a respective column of thru-passages 48 in the grid array. In this manner, a particular thru-passage 48 of the positional aid device 100 can be indicated by a combination of one of indicia 88 and one of indicia 86 such as “F4”, wherein such an indication would identify the thru-passage at the intersection of the column F and row 4. In discussions hereinbelow wherein an MRI system or device controlling an MRI scanner is configured to determine a thru-passage or thru-passages suitable for the conduct of a procedure, the MRI system or device may be configured to display alphanumeric text on an electronic display (e.g. “F4”) corresponding to the indicia of the positional aid device 100, e.g. indicia 86 and indicia 88.

The device 80 additional includes device identification indicia 90, which can serve to identify a property of device 80, for example a source, model number, compatibility, or other property of device 80. Indicia 90 may be MRI visible, for example provided by MRI visible material contained in correspondingly shaped cavities within the layer structure 22, and/or may be visible to the naked eye of a user, for example ink stamped on or embossed in or on the surface of the layer structure 22. Indicia 90 may take any suitable form and may include alphanumeric characters, dot codes, bar codes, or other patterns (including one or more asymmetric patterns) that can be recognized by the user and/or the MRI apparatus and correlated to properties of the device 90, for example properties stored in computer memory of the MRI apparatus and which can be used to facilitate a medical procedure as discussed further below.

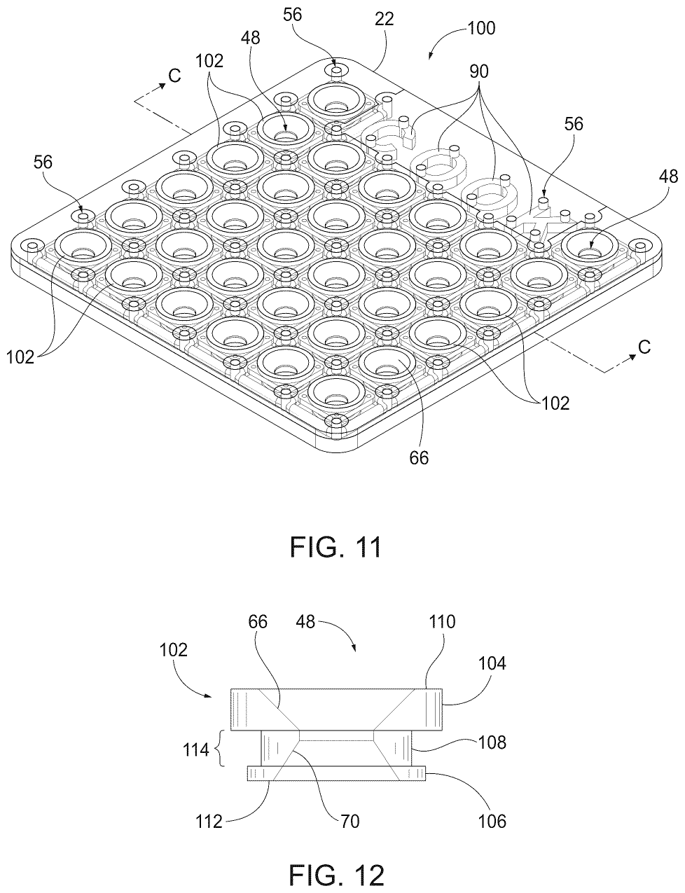

With reference now to FIGS. 11-14, illustrated is yet another embodiment of a positional aid device 100 of the present disclosure. Positional aid device 100 can have features similar to those of devices 20 and 80 discussed herein except as apparent from the figures and descriptions herein. In device 100, the ports 56 providing communication with the internal passageway system are at the first surface 28 and second surface 30 of device 100. Also, in device 100, the flexible layer structure 22 includes a plurality of rigid members 102 attached to the structure defined by the polymeric matrix 53, where the matrix material 103, for example polymeric material, of the member 102 is more rigid than the polymeric material of the polymeric matrix 53. The rigid members 102 in the illustrated embodiment each provide a respective thru-passage 48 and provide protective covers to the thru-passages 48 at both sides 24 and 26 of the device 100. The rigid member 102 can be attached to and within corresponding passages of a substrate formed of the less rigid polymeric matrix 53. Such attachment may be achieved by any suitable means including as examples in-molding the members 102 while molding the polymeric matrix 53 around them, by resiliently receiving and friction fitting the members 102 within passages of the substrate formed of the less rigid polymeric matrix 53, and/or by use of an adhesive.

With reference more particularly to FIG. 12, which provides an elevational side view of a rigid member 102, each of the rigid members 102 defines a thru-passage that provides characteristic walls (e.g. 66 and 70) and configurations of the thru-passages 48 as discussed hereinabove. The rigid member 102 in the illustrated embodiment includes a first cylindrical portion 104, a second cylindrical portion 106, and a third cylindrical portion 108 intermediate the first and second cylindrical portions 104 and 106. Third cylindrical portion 108 has an outer diameter that is smaller than that of portions 104 and 106. Second cylindrical 106 portion has an outer diameter that is small than that of portion 104. The combination of the first, second and third cylindrical portions thereby defines an annular groove 114, into which can be received volumes of the polymeric matrix 53 so as to positionally secure the member 102 with respect to the substrate formed by the polymeric matrix 53. The rigid member 102 has a first surface 110 that can provide a portion of the first surface 28 of the device 100 and a second surface 112 opposite the first surface 110 that can provide a portion of the second surface 30 of the device 100. It will be understood that while rigid members 102 in the illustrated embodiment are configured so as to provide protective covers to the thru-passages 48 at the first and second sides 24 and 26 of the device 100, in other embodiments rigid members could be configured to provide protective covers at only the first side 24 or the second side 26. Devices 100 that have protective covers to the thru-passages at least at the first side 24 will be beneficial in that the covers, made of a more rigid material, can prevent penetration of a tissue penetrating instrument such as a needle into the material of the device 100 (whereas the polymer matrix 53 material may not be sufficiently hard or rigid to do so).

In addition to providing features of thru-passages 48 and providing protective covers, the rigid members 102 can modulate the flexibility of the overall device 100, for example providing a device 100 that is less flexible than one made entirely of the polymer matrix 53 material. For example, the selection of polymeric material and the configuration of the rigid members 102 can be controlled to provide this attenuation of flexibility of the device 100. In other forms, the rigid members 102 can incorporate an MRI visible material that demarks the thru-passages 48, in addition to or as an alternative to MRI visible material incorporated elsewhere in the device 100 as discussed herein.

Any suitable materials of construction may be used to make the positional aid devices described herein. In some forms, the polymer matrix 53 will be an elastomeric polymeric material, such as a thermoset or thermoplastic elastomeric polymeric material, including as examples a silicone, a polyurethane, a polystyrene, or a natural or synthetic rubber (e.g. silicone rubber). Such a material may have a Shore A hardness no greater than about 60A, and typically in the range of about 10A to about 60A, or about 10A to about 40A, and in some forms in the range of about 10A to about 20A. In some forms, the polymer matrix 103, when included, will have a Shore D hardness of at least about 5D, or at least about 20D, and typically in the range of about 20D to about 80D.

Positional aid devices and/or one or more additional devices that can be used therewith as described herein can be provided in sterile condition in medical packaging, separately or together (e.g. as a medical kit). In certain embodiments, the medical package containing the positional aid device can be a moisture-proof package, for example a moisture-proof foil package such as a foil pouch package.

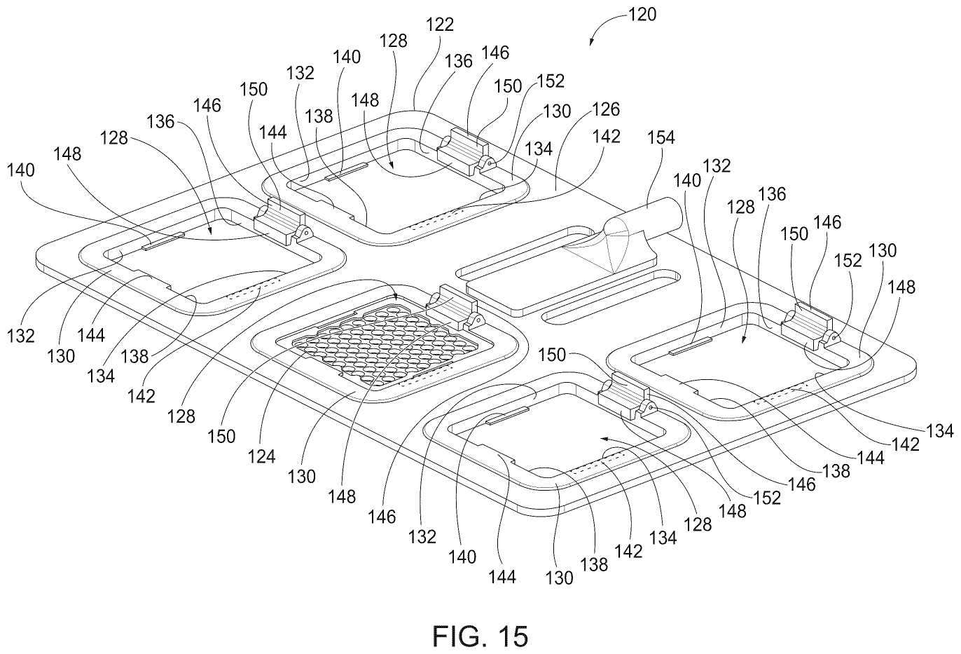

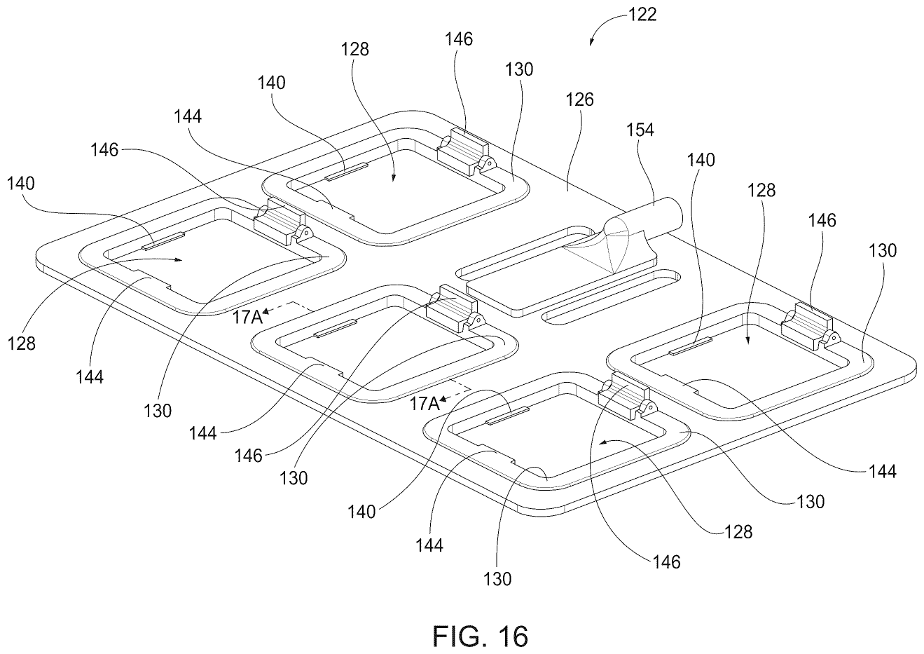

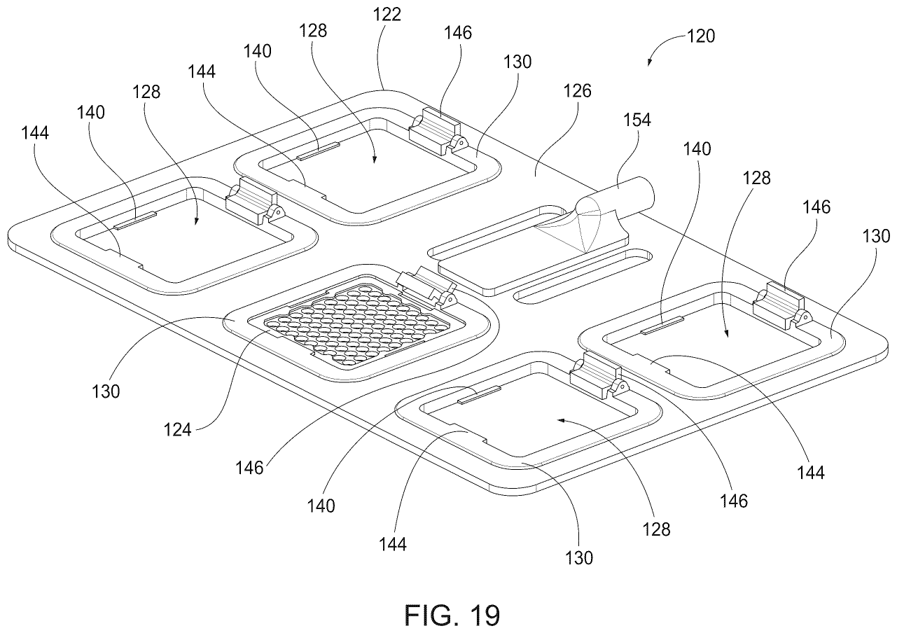

Use of the positional aid devices disclosed herein is not limited to independent use or interaction with an interventional device such as interventional device 200 discussed above. For example, the positional aid devise can be coupled with an MRI scanner component to enhance imaging during targeting and guidance by an interventional device, or with procedural equipment e.g. providing sterile access to an entry point near a region of interest, such as a medical procedure drape, or with physiological instruments for monitoring patient vitals. Examples of the MRI scanner components to which the positional aid devices may be coupled include but are not limited to MRI head, flex, or loop coils; examples of procedural equipment for coupling include but are not limited to medical procedure coverings such as drapes or blankets, or straps; and examples of physiological instruments for coupling include but are not limited to electrocardiogram leads, blood oxygen monitoring sensors, blood pressure cuffs, or temperature sensors. Of particular advantage is the incorporation of a positional aid device with an MRI coil capable of receiving scanner return signal from tissue in and around the region of interest while still providing point of entry through the skin or other outermost tissue layer by an interventional device (e.g., a needle guide device plus needle, or a needle). In one application the structure of the MRI coil can be such that it supports the positional aid device within the interior of a loop antenna of the MRI coil, or at a minimum allows access to an open area in which to directly attach the aid device to the patient's skin. One exemplary arrangement in which the structure of an MRI coil is configured to integrate a positional aid device, for example such a device as described herein, is depicted in FIGS. 15 through 19.

In particular, shown in FIGS. 15 through 19 is an MRI coil apparatus 120 including an MRI coil 122 and a positional aid device 124. Positional aid device 124 can for example be or include a positional aid device 20, 80 or 100 as described hereinabove. MRI coil 122 is preferably flexible so as to conform to a patient under the force of gravity, for example as is known for so-called “flex coil” or “surface coil” devices presently available for use in conjunction with MRI systems. MRI coil 122 includes a body 126, for example in the form of a polymeric sheet, desirably a polymeric foam sheet. The body 126 defines a plurality of openings 128. The MRI coil 122 includes a plurality of mounting frames 130 attached to the body 126, each associated with a respective opening of the plurality of openings 128. The mounting frame(s) 130 can be formed of a flexible, rigid, or semi-rigid polymeric material, in various forms. It will be understood that the body 126 in other forms may define only a single opening and include only a single mounting frame 130, or any suitable number of openings such as from one to ten openings, some or each of which may be associated with a respective mounting frame 130. The mounting frame(s) 130 each define a first internal wall 132, a second internal wall 134 opposite the first internal wall 132, a third internal wall 136, and a fourth internal wall 138 opposite the third internal wall 136. The internal walls 132, 134, 136 and 138 each present a surface facing the respective opening 128 with which the mounting frame 130 is associated.

The mounting frame(s) 130 are configured to receive and hold a positional aid device in an opening of the frame(s) aligned with opening 128, such as positional aid device 124. For these purposes the illustrate mount frame includes a first shoulder wall 140 extending transversely from first internal wall 132 in a direction into opening 128 and a second shoulder wall 142 extending transversely from second internal wall 134 in a direction into opening 128. The mount frame 30 also includes a third shoulder wall 144 extending transversely from fourth internal wall 138. As shown, the shoulder walls 140, 142 and 144 each have a thickness that is less than the height of the respective wall 132, 134, 138 from which it extends, considered in a direction perpendicular to the outer face of body 126. As well, the shoulder walls 140 and 142 are positioned at or adjacent the bottom of walls 132 and 134, whereas the shoulder wall 144 is positioned at or adjacent the top of wall 138. In this manner, the walls 132 and 134 can be received against and support a lower surface of positional aid device 124 to resist downward movement of device 124, and wall 136 can be received over and preferably against an upper surface of positional aid device 124 to resist upward movement of device 124. Thus, the mounting frame(s) in preferred forms secure the positional aid device against upward or downward movement in the opening 128, and it will be understood that such arrangements other than those specifically disclosed for MRI coil device 120 may also be used. The mount frame(s) 130 also include a fixation actuator 146. Fixation actuator 146 is movable between a first position in which the device 124 is fixed to MRI coil 122 and a second position in which the device 124 is unfixed from MRI coil 122, and can be separated therefrom. In the illustrated form, fixation actuator 146 includes a first projecting member 148, for example a wall as illustrated, that is extendable into the opening 128, and a second member 150, such as a wall member as illustrated, configured and positioned for manual operation by a user to extend first member 148 into opening in the first position of fixation actuator 146 and to move the first member 148 away from and potentially out of opening 128 in the second position of fixation actuator 146. The fixation actuator 146 can in some forms be pivotable between the first position and the second position, for example wherein the actuator includes a pivot pin 152 and the first and second members 148 and 150 are incorporated in a unitary member that is pivotable about the pivot pin 152. The fixation actuator 146 may for example include a latch or other projecting member that is received within a corresponding fixation opening of the positional aid device 124 (see e.g. discussions below). It will be understood that other arrangements for selectively fixing and unfixing the device 124 to the MRI coil 122 can also be used, including those that utilize one or multiple fixation actuators that move between a first position for fixing the device 124 to the coil 122 and a second position for unfixing the device 124 from the coil 122. The MRI coil 122 also includes a coil link connector 154, for communicating radiofrequency signals received by the coil 122 to an MRI scanner.

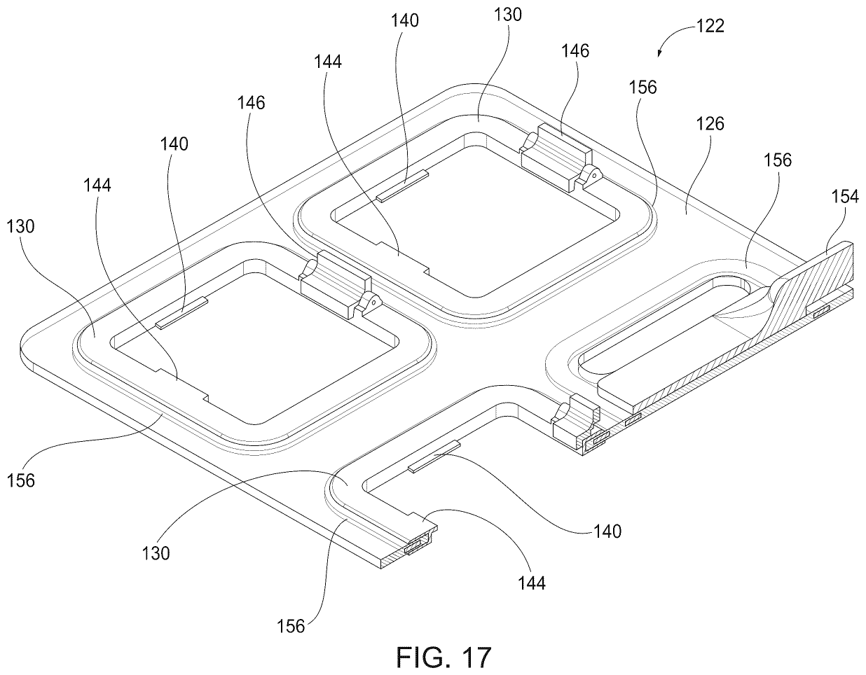

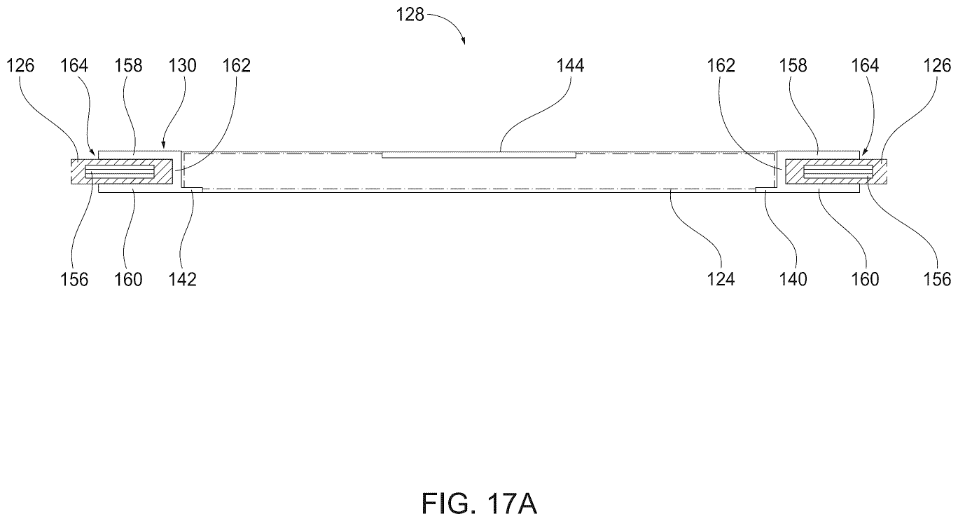

Referring now more particularly to FIGS. 17 and 17A, the MRI coil 122 also includes a plurality of loop antennas 156. Loop antennas 156 are embedded within the body 126 and surround the openings 128. Other loop antennas may also be included, that do not surround any of the openings 128, for example the loop antenna 156 shown that is associated with the coil link connector 154. The mount frame(s) 130 each include a peripheral upper wall 158 and a peripheral lower wall 160, which can extend parallel to one another as illustrated, that are connected by a transverse (e.g. perpendicular) wall 162 that in the illustrated embodiment provides internal walls 132, 134, 136 and 138 discussed above. The walls 158, 160 and 162 define a groove 164 extending about and open at the circumference of mount frame(s) 130, and portions of the body 126 are received within the groove 164 to connect the mount frame(s) to body 126, as illustrated. Additionally or alternatively, adhesive agents or other methods of bonding or attaching the mount frame(s) 130 to the body 126 may be used. As also illustrated, at least a portion of the embedded antenna(s) may also be received within the groove 164.

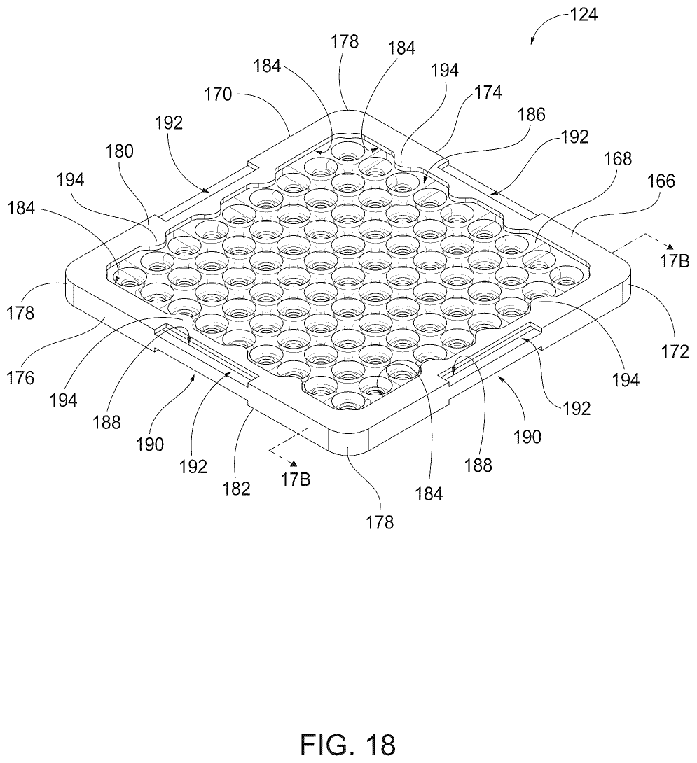

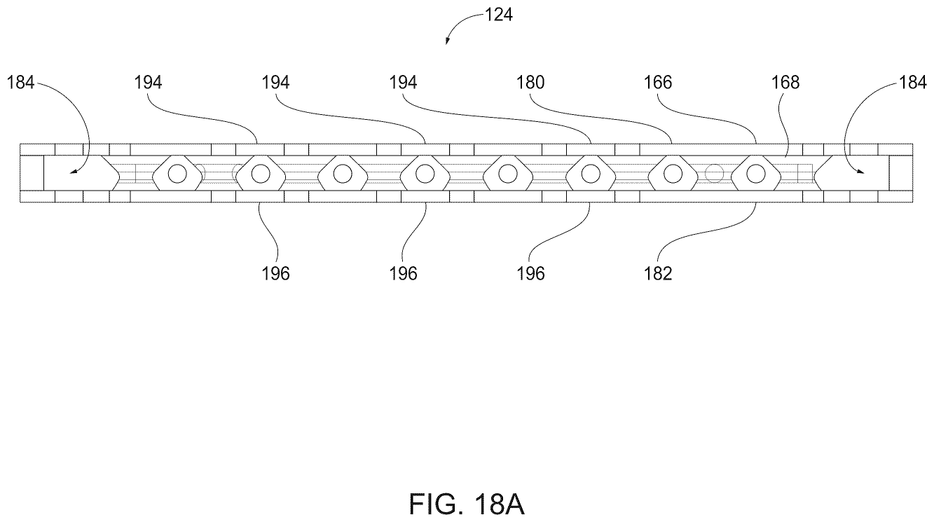

Referring now more particularly to FIGS. 18 and 18A, features of the illustrative positional aid device 124 will be described. Positional aid device 124 includes a mount housing 166, for example in the form of a peripheral frame, and a positional aid device 168 received therein. Positional aid device 168 may in some forms be a device 20, 80, or 100 as discussed hereinabove. Mount housing 166 includes a first edge wall 170, a second edge wall 172 opposite the first edge wall 170, a third edge wall 174, and a fourth edge wall 176 opposite the third edge wall 174. These edge walls may be adjoined by radiused corner edge 178, as illustrated. The mount housing 166 defines a top wall 180 and a bottom wall 182 opposite the top wall 180. The edge walls 170, 172, 174 and 176 together with the top wall 180 and the bottom wall 182 define a peripheral groove 184 that is open inwardly to the opening 186 defined by the mount housing 166. When mounted in the mount housing 166, a peripheral portion of the positional aid device 124 is received within the groove 184.

The mount housing 166 defines a plurality of fixation openings, for example slots 188, for receiving and contacting the projecting member 148 of fixation actuator 146 (see e.g. FIG. 15) in the first (fixed) position of the actuator 146. While in the illustrated form a plurality of slots 188 are provided, for example so that the device 124 can be oriented in a number of ways and still present a slot 188 for cooperating with actuator 146 to fix the positional aid device to the MRI coil device, it will understood that in other forms the mount housing 166 can include a single slot 188, at least one slot 188, or multiple slots 188. The mount housing 166 also defines a plurality of recesses 190 and 192 for receiving shoulder walls 140, 142 and 144 when device 124 is received within a mounting frame 130 of MRI coil 122. The top wall 180 of the mount housing 166 defines a plurality of projections 194 extending in the direction into opening 186 and the bottom wall 182 of the mount housing 166 defines a plurality of projections 196 extending inwardly into opening, which can facilitate a secure fit of the positional aid device 168 in the mount housing 166. Projections 194 and 196 may positionally overlap one another and/or may be in the form of convexly-curved tab members, as shown.

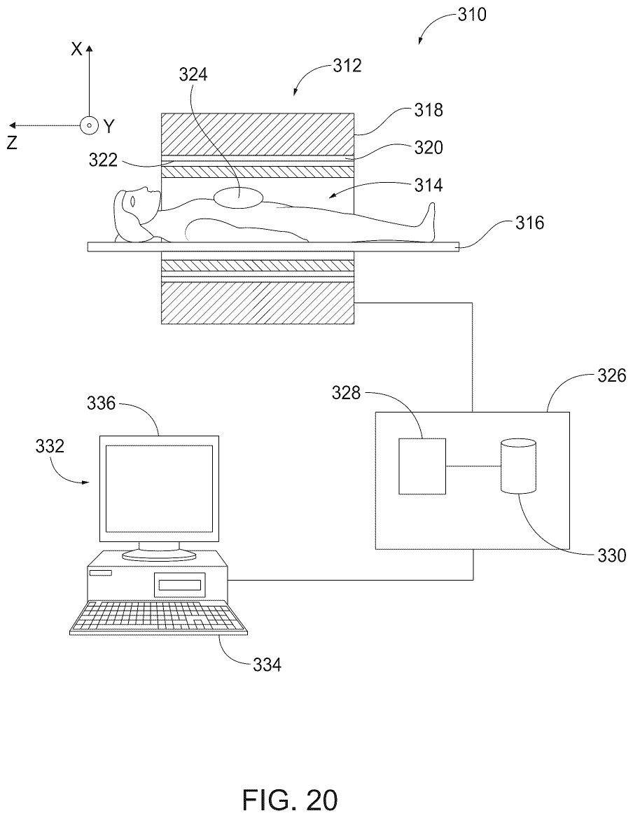

The positional aid devices disclosed herein can be used in conjunction with and/or be a component of an MRI system. In this regard, FIG. 20 provides a schematic representation of an example MRI system 310 in accordance with certain aspects of the present disclosure. The MRI system 310 includes the actual magnetic resonance scanner (data acquisition unit) 312 with an examination space or patient tunnel 314 in which a patient can be positioned on a driven bed 316.

The magnetic resonance scanner 312 is typically equipped with a basic or primary field magnet system 318, a gradient system 320, as well as an RF transmission antenna system 322 and an RF reception antenna system 324, e.g. a surface coil which can include one or more loop antennas as described herein. In certain embodiments, a positional aid device can be mounted on the surface coil, for example as discussed in connection with FIGS. 15 to 19 for MRI coil device 120, or a positional aid device can be closely associated with (e.g. placed under and against the skin of the patient, potentially adhered to the skin) the surface coil. In the shown exemplary embodiment, the RF transmission antenna system 322 is a whole-body coil permanently installed in the magnetic resonance scanner 312. However, the whole-body coil can also be used as an RF reception antenna system.

The basic field magnet system 318 typically generates a basic or primary magnetic field in the longitudinal direction of the patient, i.e. along the longitudinal axis of the magnetic resonance scanner 312 that proceeds in the z-direction. The gradient system 320 typically includes individually-controllable gradient coils to selectively switch (activate) gradients in the x-direction, y-direction, or z-direction independently of one another.

The MRI system 310 as shown is a whole-body system with a patient tunnel into which a patient can be completely introduced. However, in principle the embodiments as described herein may also be used with other MRI systems, for example with a laterally open, C-shaped housing, as well as in smaller magnetic resonance scanners in which only one body part can be positioned.

The MRI system 310 has a central control device 326 that is used to control the MRI system 310. Control device 326 typically includes at least one computer processor 328, and potentially multiple computer processors, and at least one electronic memory storage device 330, and potentially multiple such memory storage devices. As is known, the control device can include other circuitry components as well. This central control device 326 is configured to control a series of radio-frequency pulses (RF pulses) and gradient pulses depending on a selected pulse sequence or, respectively, a series of multiple pulse sequence to acquire magnetic resonance images of slices of the scanned region. For example, such a series of pulse sequence can be predetermined. Different control protocols for different scan sessions are typically stored in memory 330 and can be selected by an operator and potentially modified as needed or desired.

Operation of the central control device 326 can take place via a terminal 332, which includes a user input device 334 and an electronic display 336 for such a purpose, through which the entire MRI system 310 can thus also be operated by a user. MR images can also be displayed at the display 336, and scan sessions can be planned and started by means of the input device 334 potentially in combination with the electronic display 336. Moreover, suitable control protocols may be selected (and possibly modified) with a suitable series of pulse sequences. Additionally, in typical forms, the MRI system 310 will also include another electronic display or displays (additional to display 336) positioned in the vicinity of the scanner, e.g. for viewing by a clinician or other health care working performing a procedure on a patient as guided by the MRI system.

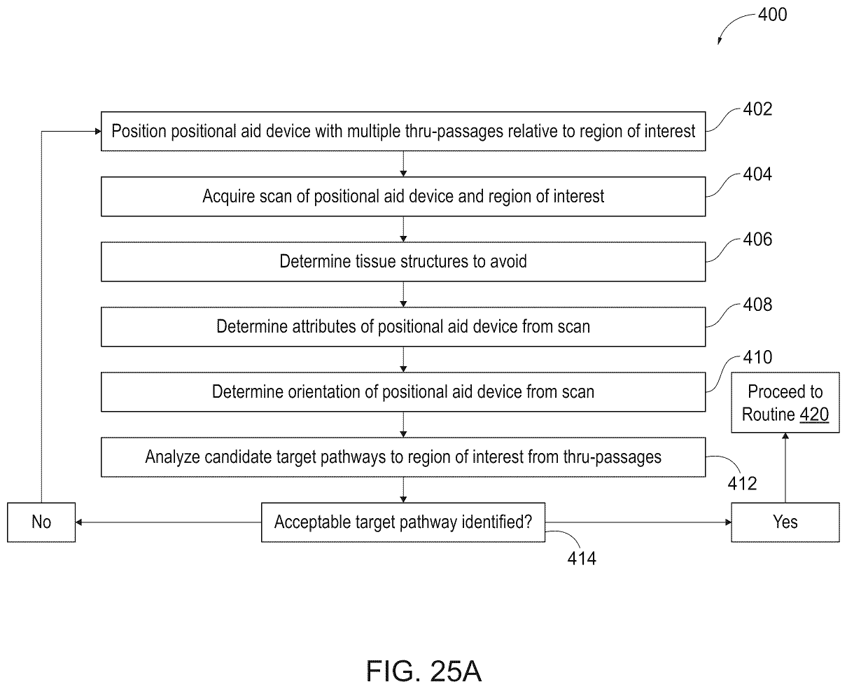

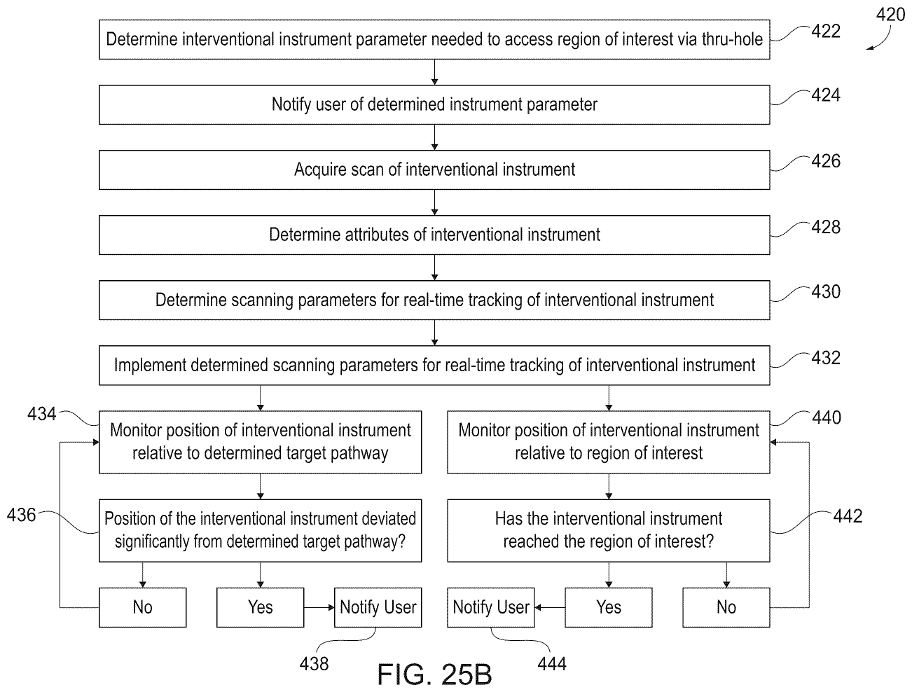

In certain embodiments herein, the control unit 326 can be configured to perform methods and method steps according to the present disclosure, including for example those discussed in conjunction with the Exemplary Uses and/or FIG. 25A and/or FIG. 25B below. Such configuration of the control unit 326 may be implemented as hardware (e.g. computer processors), software, or a combination of both hardware and software (e.g. a non-transitory computer-readable medium with executable instructions stored thereon). It will be understood that the control unit 326 may include additional or alternate components as well. The manner by which suitable raw data are acquired by radiation of RF pulses, the generation of gradient fields, and MR images are reconstructed from the raw data, may be performed in any suitable manner, such as using known techniques, and thus need not be explained in detail herein.

### EXEMPLARY USES OF THE DISCLOSED POSITIONAL AID DEVICES

Positional aid devices as described herein may be used in one or multiple ways to facilitate an image-guided interventional medical procedure on a patient.

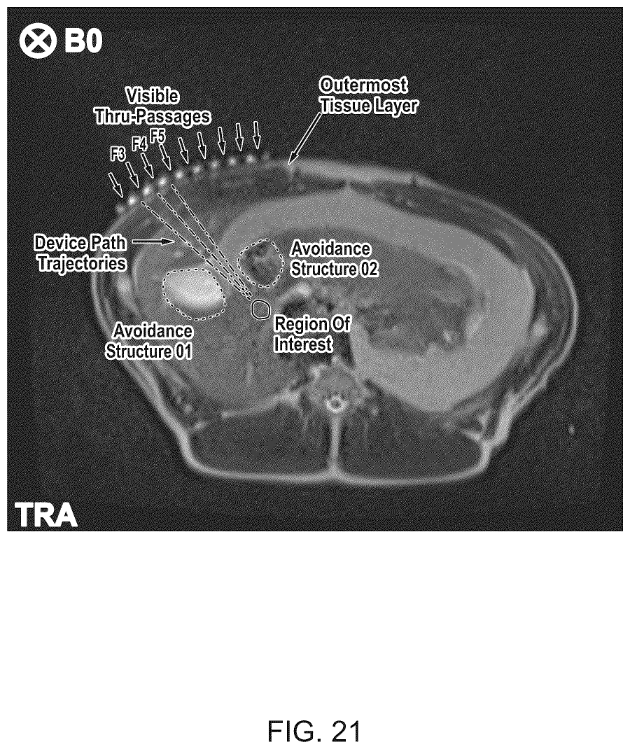

As one illustrative example, FIG. 21 depicts an MRI image with annotations illustrating aspects of a use of a positional aid device (e.g. device 20, 80 or 100 discussed hereinabove) to position and orientate an interventional device (e.g., biopsy needle) for targeting a region of interest within a patient's body. In this process the positional aid device is first positioned over, for example adhered onto, the skin or other outermost tissue layer of the patient's body and over the region of interest within the body. The patient is then scanned under magnetic resonance imaging to identify potential device path trajectories to a region of interest, as annotated, from a selected one or selected set of the thru-passages of the positional aid device (annotated in FIG. 21 with alphanumeric indicia “F3”, F4″, and “F5”, indicating the thru-passages identified by column (F) and row (3, 4, or 5) of visible indicia of the positional aid device (see e.g. FIG. 10 and discussions thereof). Factors in evaluating these potential trajectories include the avoidance of critical tissues, structures, or vasculature enroute to the region of interest, and the region of interest's depth below the skin from each point of entry (i.e., depth from the skin area positioned under each thru-passage of the positional aid device). After selecting a trajectory, the corresponding thru-passage for entry on the positional aid device is identified, for instance by the physician or other user. From there the physician/user may use any one of three options to target the region of interest with an interventional device. A first option includes marking the patient's skin through the thru-passage at the point of entry and then the removal the positional aid device. Targeting of the region of interest with the interventional device is then done without the aid of the positional aid device, either manually or under some other form of guidance. A second option includes targeting the region of interest with the positional aid device in-place either manually or under some form of guidance that does not directly interface with the positional aid device. Finally, a third option utilizes an interventional device with at least one surface that conforms to or interfaces with the outward facing surface of the positional aid device, e.g. a surface surrounding a thru-passage of positional aid device, and can be used in conjunction with the aid device to facilitate targeting the region of interest (see e.g. FIGS. 5 through 9 and related discussions). With this option the outward facing surface of the positional aid device is used to position the interventional device at the desired entry point of the patient's skin with respect to the region of interest, and contact between such outward facing surface of the positional aid and the surface of the interventional tool is used to stabilize the interventional tool while it is moved, for example pivoted, to align its longitudinal axis with the selected path trajectory.

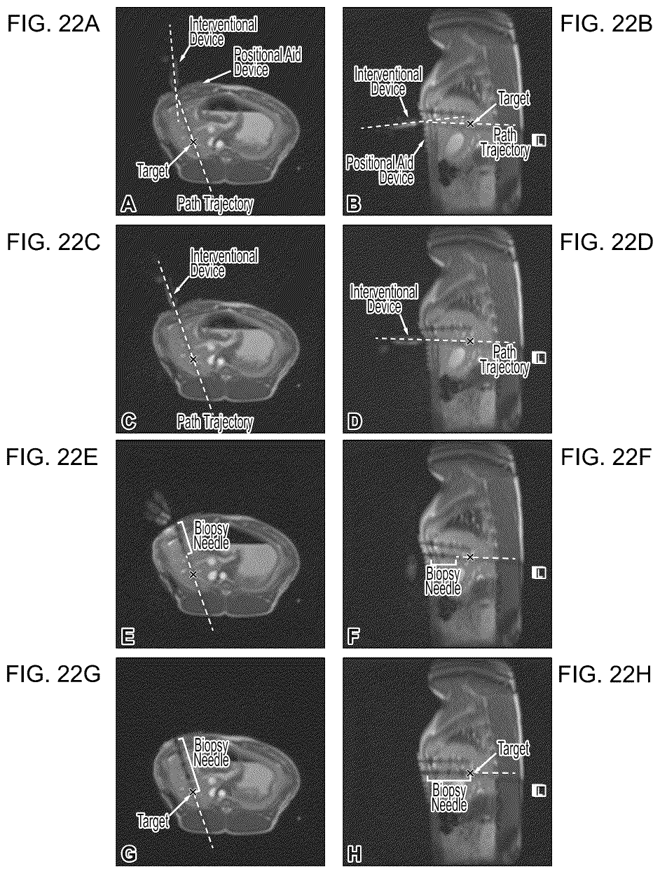

FIGS. 22A through 22H are sequential MRI images that illustrate a version of the third option discussed above under live magnetic resonance imaging guidance utilizing two orthogonal bi-plane images. In FIGS. 22A and 22B the interventional device is shown interfacing with the positional aid device at the thru-passage defining the entry point through the outermost tissue layer (skin), but with its longitudinal axis slightly misaligned from the path trajectory to the target region of interest. In FIGS. 22C and 22D, the interventional device has been pivoted with its distal surface in contact with the outward-facing contour of the surface surrounding a selected thru-passage to longitudinal alignment with the desired path trajectory, and the biopsy needle advanced into the tissue proximal of the target region. FIGS. 22E and 22F depict the advancement of the biopsy needle along the path trajectory with the interventional device removed, but the positional aid device still in place. Finally, FIGS. 22G and 22H show the distal tip of the biopsy needle reaching the target region.

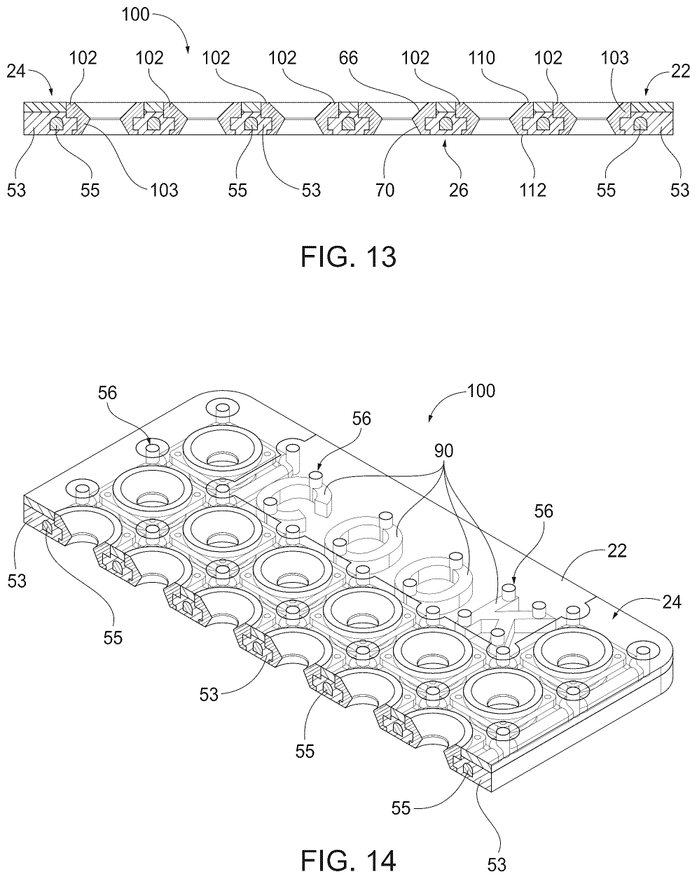

Several methods can be used to automatically detect the position and orientation of the positional aid device under magnetic resonance imaging. Methods include the identification of distinct imaging features originating from the signal generated by internal passages as viewed from the outward facing side of the positional aid device (e.g., internal passages 55 as viewed from surface 24 of FIG. 1), and in the patterning of filled internal passages as viewed from a cross section of the aid device under general and specific magnetic resonance imaging modalities (passages 55 as viewed from cross section in FIG. 2; also see FIG. 20). In these instances, asymmetries in geometry of passages or changes in geometry could aid in the auto-recognition of the positional aid device as well as allow for the determination of the device's orientation. In the first instance, distinct shapes in the indicia extending along the axes of the aid device (e.g. components 86 and 88 of FIG. 10) and in any identification indicia (e.g. component 90 of FIG. 10) could be used for pattern recognition by computation image processing and machine learning algorithms (i.e., artificial intelligence) to detect the presence of the positional aid device. Since these indicia reside at particular locations with respect to the rest of the positional aid device, the imaging attributes they create can allow for the determination of device orientation. Alternatively, changes in the image attributes stemming from internal passages surrounding thru-passages as viewed from different cross-sectional angles could be used to determine the positional aid device's orientation, with the outermost passage marking the boundary of the device. For instance, the intersection of different cross-sectional views not parallel with the square internal passage pattern from FIGS. 11 to 14 would yield different spacing between the individual sections of MRI visible material (component 55 of FIG. 13). Based on this spacing, the orientation of the aid device could be determined. Finally, a combination of any of these identification schemes with specific imaging modalities that highlight the image attributes over the rest of the device or surrounding tissue could further aid in auto-detection and orientation of the positional aid device within the scanner.

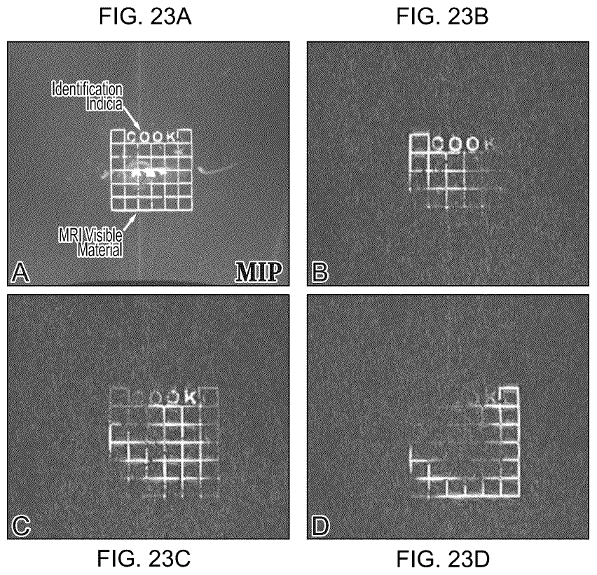

FIG. 23A demonstrates one such approach where a maximum intensity projection (or MIP) of the device's MRI visible material as obtained from multiple evenly-spaced parallel imaging planes is projected onto a single two-dimensional plane. With this projected image enabling the detection and orientation of the aid device in two-dimensional space, the series of parallel imaging planes used to construct the MIP (or a new series of imaging planes perpendicular to the projected plane) can then be used to determine out-of-plane deviation from the MIP image. For example, FIGS. 23B to 23D depict a series of three parallel imaging planes used to construct the single projected MIP image shown in FIG. 23A. As shown in FIGS. 23B to 23D, the intensity of signal generated by the MRI visible material varies across planes with higher intensity indicating a larger quantity of visible material residing on that plane. Since parallel imaging planes are evenly spaced, the image intensity of a portion of internal passage can be correlated to the plane in which it resides and used to determine the contour of the positional aid in three-dimensional space. Utilizing data of this third dimensional component along with the two-dimensional orientation, the full contour of the positional aid device can be estimated.

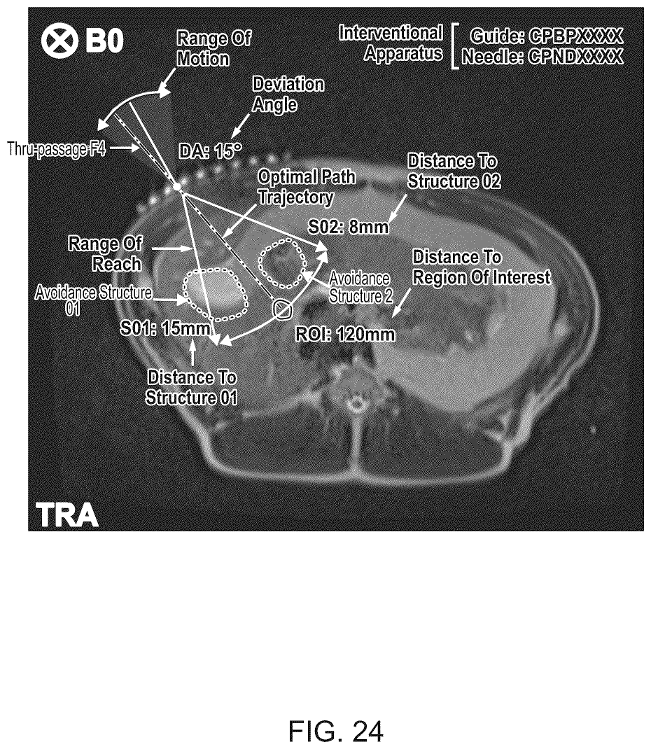

With the position and orientation of the positional aid device fully determined (via autodetection), the navigation and targeting of a region of interest as depicted in FIG. 21 can at least be partially automated, as for example illustrated in conjunction with FIG. 24. After detection, attributes of the positional aid device can be applied to a model that is then registered with the imaging features distinguishing the aid device from the surrounding tissue. These attributes could include but are not limited to the overall dimensions of the device, the arrangement and location of all thru-passages, the range of motion of an interventional device interfacing with the positional aid device as defined by the contour of the thru-passage walls on the aid device's outward or user-facing surface (range of motion 74, thru-passage walls 66, and outward facing surface 24 of FIG. 5), the range of reach of an interventional apparatus interfacing with the positional aid device as defined by the contour of the thru-passage walls on the aid device's inward or patient-facing surface (range of motion 76, thru-passage walls 70, and outward facing surface 26 of FIG. 5), imaging sequences that promote visualization of the aid device or any anatomy relevant to the procedure, enable automation protocols for the use of the aid device during a procedure, and queue auto-detection algorithms for ancillary devices interfacing with the positional aid device (e.g., interventional device 200 of FIG. 6). Utilizing these features, device path trajectories emanating from each thru-passage of the positional aid device can be projected into a patient's body and used to determine the optimal approach to reach a region of interest with avoidance of critical tissues, structures, or vasculature enroute to the region. This process as described is not limited to the series of imaging planes as defined by the MRI operator to localize the positional aid device (or region of interest); instead, a new series of planes, either aligned with the axes (or alternative feature) of the aid device or otherwise, can be automatically selected by software considering the relative position of the positional aid device, region of interest, and any obstructive anatomy in three-dimensional space. From the determination of these relative locations and optimal path trajectories, a recommended set of interventional apparatus (e.g., guide device and needle) can be provided to the physician or other user or automatically selected using software. Finally, the detection and localization of the positional aid device, optimal path trajectories, etc., can be determined recursively to compensate for the movement of devices and/or anatomy throughout the procedure (e.g., as a result of breathing) and visual feed queues and metrics (e.g., deviation angle from path trajectory, distance to region of interest, distance from avoidance anatomy, etc.) can be overlayed over the image to aid the physician in targeting the region of interest. FIG. 24 is annotated to show an optimal determined target pathway, distances separating the determined target pathway (optimal path trajectory), determined tissue structures to be avoided, a distance to the region of interest from the skin or other outermost tissue layer of the patient, minimum distances between the tissue structures to be avoided and the determined target pathway, a range of angles and distal reaches available for insertion of a selected interventional instrument (e.g. needle) through a selected thru-passage of a positional aid device, and a deviation angle of the determined target path from the longitudinal axis of the selected thru-passage, and an alphanumerical annotation identifying the visible indicia of the positional aid device that correlate to the selected thru-passage (i.e. “F4”, with the letter F and the number 4 corresponding respectively to column F and row 4 of the positional aid device, such as that in FIG. 10). In aspects herein, the MRI system can be configured to overlay one or more graphics on MRI images displayed by the system representing at least one, at least some, or all of these annotated features of FIG. 24, or of FIG. 21 discussed above.

While discussions above refer to steps involving the use of a positional aid device as disclosed herein in conjunction with an MRI scanner or system, it will be understood that such steps may also be conducted with devices or apparatuses herein that incorporate the positional aid device along with another device or structure, including for example the positional aid device as mounted in or otherwise attached to a surface MRI coil as discussed hereinabove in conjunction with FIGS. 15 to 19, or as attached to a medical procedure drape covering the patient, or otherwise.

FIGS. 25A and 25B are schematics that illustrate various steps and combinations of steps that can be incorporated in methods and/or in device or system configurations herein. FIG. 25A illustrates a routine 400 including a series of steps that can be conducted prior to inserting an interventional instrument into a patient. In step 402, a positional aid device including multiple through passages, for example any of those discussed herein such as device 20, 80 or 100 (individually or incorporated with another device structure, e.g. as in MRI coil device 120), can be positioned adjacent a patient, for example against the skin, relative to a tissue region of interest within the patient. This step will typically be performed manually by a clinician or other health care worker user. In step 404, an MRI scan is acquired that includes the positional aid device and the region of interest. In step 406, tissue structures to be avoided when inserting an interventional device into the patient and advancing the interventional device to the region of interest are identified. This too can be performed by the MRI system using a computer processor and acquired MRI scan data, potentially with user input on a user input device (e.g. keyboard device and/or electronic touch screen display device) to remove, modify and/or add to the tissue structure(s) for avoidance identified by the MRI system. This MRI scan can generate image data processed and displayed on an electronic display device. In step 408, the data from the MRI scan, including data acquired as a result of the presence of the MRI visible material in the positional aid device, is analyzed to determine attributes of the positional aid device. Such attributes may, for example, include a commercial source, a make and/or model, dimensional characteristics, number and relative positions of thru-passages, the available range of motion for an instrument (e.g. angles of insertion) associated with the thru-passages (e.g. as discussed hereinabove for passages of devices 20, 80 and 100; see particularly FIG. 5 and discussions thereof), and/or other attributes. In some forms, these attributes are determined directly from the scan data. In other forms, an identifying feature on the positional aid device, for example recognizable by the MRI system due to the presence of the MRI visible material therein, can provide an identification of the positional aid device by which the MRI system, using a computer processor, can look up these attributes of the positional aid device stored in electronic memory, for example in a lookup table stored in the memory. In step 410, the orientation of the positional aid device, including for example its rotational orientation and/or its three-dimensional (3D) conformational shape, is determined. For example, rotational orientation can be determined by the MRI system using a computer processor and scan data from an MRI-detectable asymmetric feature of the positional aid device, for example an asymmetric feature defined by MRI visible material incorporated within the positional aid device as discussed hereinabove. 3D conformational shape of the positional aid device can be determined by the MRI system using a computer processor and acquired MRI scan data, for example using steps as discussed above in connection with FIGS. 23A through 23D. In step 412, the MRI system analyzes multiple candidate target pathways (sometimes herein also called “path trajectories”) between multiple thru-passages of the positional aid device and the region of interest and then in step 414 determines whether an acceptable target pathway is available from one or more of the thru-passages. Such an analysis can be based at least in part on the determined attributes of the positional aid device, for example the available range of instrument motion (e.g. angles of instrument insertion) through the thru-passages as discussed above. As well, the thru-passages included in the analysis can be determined by the MRI system based on scan data, potentially utilizing imaging processing software, or in other forms a user can identify and input thru-passage(s) to be included in the analysis whereupon the MRI system is configured to analyze the input thru-passage(s) to determine whether an acceptable target pathway is available from one or more of them. It is advantageous in embodiments herein that thru-passages of the positional aid device provide a range of angles for instrument insertion, as this increases the likelihood that an acceptable target pathway to the region of interest will be determinable from one or more of the thru-passages. The MRI system can then notify the user of such one or more acceptable target pathways if available, and can potentially be configured such that the user can confirm an identified target pathway for utilization in the procedure through a command on a user input device. If the MRI system determines in step 414 that there are no viable target pathways from any thru-passage of the positional aid device to the region of interest while avoiding tissue structures identified for avoidance, the system notifies the user of the same, for example visually on an electronic display including for example by displaying a written message and/or a recognizable icon and/or audibly by a speaker component of the MRI system. The user may potentially then reposition the positional aid device (repeating step 402), and steps 404, 406, 410, and 412 and potentially also step 408 can be repeated until an acceptable target pathway has been identified. In some forms, the MRI system can be configured to determine and notify the user of a suggested direction and/or distance of movement of the positional aid device based on the acquired scanner data and determination(s) in the previous iteration of steps of the routine 400, to facilitate such a repositioning step. Following this, an MRI-guided interventional procedure may proceed, for example a procedure including a routine 420 as illustrated in FIG. 25B.

FIG. 25B sets forth steps of an illustrative routine 420 that involves the use of an interventional instrument to access a region of interest, for example as an addition to routine 400 of FIG. 25A. In step 422, the MRI system determines an interventional instrument parameter needed to access the region of interest. The parameter can relate to a dimensional characteristic of the interventional instrument, such as the length, a diameter and/or shape of the instrument (e.g. biopsy needle or other needle, e.g. optionally as incorporated in an interventional device 200 as discussed hereinabove). The MRI system can make such a determination by analyzing with a computer processor acquired MRI scan data related to tissue structures identified for avoidance and/or to the length of the selected target path from a thru-passage of the positional aid device to the region of interest, as examples. In step 424, the MRI system can notify a user of the determined instrument parameter, for example visually on an electronic display screen and/or audibly through a speaker. A user can then initiate use of the interventional instrument in the procedure. When the instrument is moved into the scannable region, in step 426, an MRI scan of the instrument can be acquired. Based on this acquired scan data, one or multiple attributes of the instrument may be determined in step 428. The attributes are in some aspects herein determined directly from the scan data. In other aspects, an identifying feature on the interventional instrument, for example recognizable by the MRI system due to the presence of an MRI visible material therein and/or due to the presence of a pattern of MRI markers, such as passive MRI artifact markers, can provide an identification of the positional aid device by which the MRI system, using a computer processor, can look up attributes of the positional aid device stored in electronic memory, for example in a lookup table stored in the memory. In addition or alternatively, a user can input an identifier (e.g. make/model number) of the interventional instrument to the system via a user input device and a computer processor can use the identifier to look up attributes of the interventional device stored in electronic memory of the MRI system. The attributes stored in electronic memory and/or otherwise determined may include, for example, a limited range of motion available (e.g. limited range of path trajectory angles available) when using the thru-passages of the positional aid device to introduce the interventional device into a patient, for example as described in connection with FIG. 5 hereinabove. In step 430, the MRI system can determine MRI scanning parameters, such as sequence parameters, for real-time tracking and/or imaging of the interventional instrument, based on the determined attributes of the instrument. For example, the scanning parameters may be stored in electronic memory and correlated to the attribute(s) of the interventional device, and a computer processor may look up the scanning parameters based on the instrument attribute(s) either detected by the MRI system using scan data or input by a user. In step 432, the determined scanning parameters are implemented for real-time tracking, typically including real-time imaging, of the interventional instrument during the procedure.

In step 434, during the real-time tracking, the position of the interventional instrument relative to the determined target pathway is monitored as the instrument is moved, e.g. into engagement with the positional aid device and/or toward the tissue region of interest. During the monitoring of step 434, if the interventional instrument is engaged at a thru-passage of the positional aid device other than one that has been determined acceptable or has been selected for the procedure, the MRI system can notify a user of that occurrence, for example by a visual notification on an electronic display screen and/or by an audible notification through a speaker of the system. In step 436, if the position of the interventional instrument while within patient tissue has deviated significantly from the determined target pathway, the MRI system can notify the user in step 438, for example by a visible notification on an electronic display screen and/or by an audible notification through a speaker of the system. The user can then determine an action to be taken, for example partially or completely withdrawing the interventional instrument away from the region of interest and re-directing it toward or onto the determined target pathway. The position of the instrument can of course be monitored by the system during these actions as well. On the other hand, if the position of the interventional instrument has not deviated significantly from the determined target pathway, the MRI system can continue to monitor the position of the instrument as it is moved through patient tissue toward the region of interest.

In step 440, during the real-time tracking, the position of the interventional instrument relative to the region of interest is monitored as the instrument is moved toward the region of interest, using a computer processor and image data from the MRI scanner. The MRI system may be configured to, during this monitoring step, determine and provide notice in real time to a user related to the extent of travel of the interventional instrument, for example including real time determination and notice of the remaining distance of travel of the interventional instrument required to reach the tissue region of interest. Such notice may for example be in the form of a numerical value visibly displayed on an electronic display of the MRI system that is adjusted in real time as the interventional instrument is moved relative to (e.g. toward) the region of interest. In step 442, the MRI system determines whether the instrument has reached the tissue region of interest (e.g. with its distal region therein). If the interventional instrument has not reached the region of interest (e.g. with its distal region therein), the MRI system can continue to monitor the position of the instrument relative to the region of interest as it is moved toward the region of interest (repeat step 440). If the interventional instrument has reached the region of interest (e.g. with its distal region therein), the MRI system can notify the user in step 444, for example by a visible notification on an electronic display screen and/or by an audible notification through a speaker of the system. If appropriate, the user can then continue with a procedural action in the region of interest using the interventional instrument, for example taking a biopsy where the instrument is a biopsy needle, or ablating tissue where the instrument is an ablation instrument, or delivering a flowable diagnostic or therapeutic agent to the region of interest where the instrument is an infusion needle having a lumen for carrying the flowable agent.

It will be understood that the MRI systems discussed in conjunction with FIG. 20 hereinabove can be configured to perform the steps of routines 400 and 420 of FIGS. 25A and 25B using one or more computer processors thereof, and other steps herein, unless those steps are expressly discussed as limited to manual steps conducted by a user. In this regard, the corresponding components discussed in respect of the MRI systems of FIG. 20 can be employed in the implementation of these routines 400 and 420 (e.g. electronic memory components, computer processor(s), user input device(s), electronic displays, etc. As well, it will be understood that the scan acquisition steps of the routines 400 and 420 can generate data that is processed into an image, or multiple images, displayed on an electronic display device and visible to the user. Also, it will be understood that although the above discussions refer to steps or processes conducted by an “MRI system” configured to do so, corresponding embodiments herein relate to controller devices for controlling an MRI system (e.g. central control device 326 and/or terminal 332 of FIG. 20) that do not necessarily include an MRI scanner but are configured to control an MRI scanner (e.g. scanner 312 of FIG. 20). It will further be understood that other MRI scans of the region of interest may be available to the user either acquired in the same procedure or in some cases acquired in a previous procedure, for example a previous diagnostic procedure. These and other variations will be apparent to those skilled in the art from the descriptions herein.

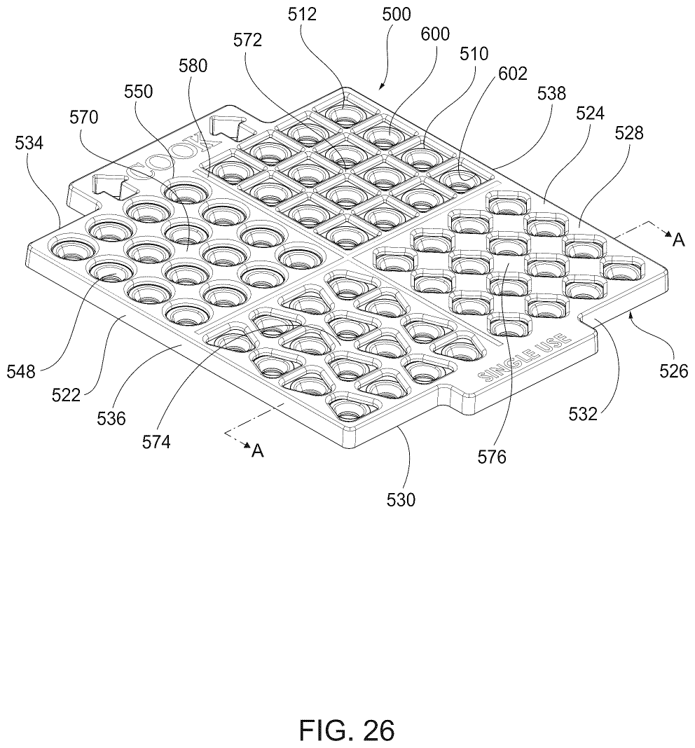

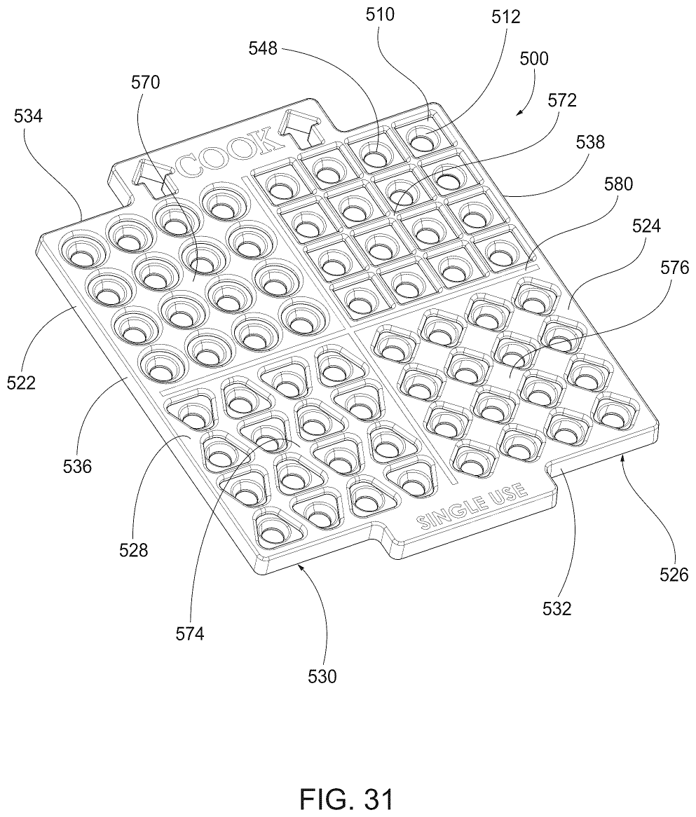

With reference to those embodiments shown in FIGS. 26 through 29b and FIG. 31, certain embodiments of a positional aid device 500 are shown. Positional aid device 500 is at least partially visible under MRI. Positional aid device 500 includes a flexible layer structure 522. The flexible layer structure can comprise one or more flexible materials incorporated such that the positional aid device 500 can conform to a curvilinear surface such as a patient tissue surface. The flexible layer structure 522 can incorporate an MRI visible material in any suitable way. In certain embodiments, flexible layer structure 522 comprises an elastomeric polymeric material, such as a thermoset or thermoplastic elastomeric polymeric material. In accordance with some forms, the elastomeric polymeric material comprises a silicone (e.g. polysiloxane). In certain embodiments the silicone is platinum cured, meaning the silicone has been reacted with a hydride in the presence of a platinum catalyst. including as examples a (e.g. silicone rubber). In preferred embodiments the flexible layer structure 522 comprises an elastomeric polymeric material, such as silicone, that is visible under MRI. In other forms, the elastomeric or other polymeric material of the flexible layer structure may be impregnated with another material that renders it visible under MRI, e.g. as discussed above.

As shown, flexible layer structure 522 of the illustrated embodiments includes a first side 524 opposite a second side 526. In use, the first side 524 typically faces away from the underlying patient tissue and towards a user of the positional aid device 500. The first side 524 defines a first surface 528. As discussed further herein, the first surface 528 can have features configured to facilitate medical procedures, such as in-bore percutaneous interventions. The second side 526 is configured to face toward patient tissue, such as skin. The second side 526 defines a second surface 530. In one version, the second surface 530 includes an adhesive surface or layer (not shown) on all or a portion thereof configured to temporarily maintain the positional aid device 500 at a fixed position on patient tissue. The adhesive surface or layer may include any suitable configuration or adhesive. The adhesive may for example be a pressure-sensitive adhesive, and in some forms may be a dry adhesive configuration to the surface that utilizes Van der Waal interactions that promote surface adhesion.

The flexible layer structure 500 of the illustrated embodiment further defines a first edge 532, a second edge 534 opposite the first edge 532, a third edge 536, and a fourth edge 538 opposite the third edge 536. As will be appreciated, in the illustrated embodiment each of the first edge 532, the second edge 534, the third edge 536, and the fourth edge 538 extends between and connects the first surface 528 and the second surface 530. According to one example, the flexible layer structure 522 has a substantially constant thickness and the first edge 532, the second edge 534, the third edge 536, and the fourth edge 538 all have a same height dimension. In some forms, the flexible layer structure 522 has a thickness of less than about 2 centimeters, or less than about 1 centimeter; in addition, or alternatively, the flexible layer structure 22 can have a thickness of at least about 0.2 centimeters, or at least about 0.5 centimeters. Embodiments wherein the flexible layer structure 522 has a thickness of at least about 0.5 centimeters will be particularly beneficial in configurations constructed for interaction with other medical instruments, e.g. as described herein, as they can provide significant surfaces extending transverse to the first surface 528 and/or the second surface 530 for abutment against and interaction with the instruments (e.g. to stabilize a position and/or limit a range of motion thereof relative to the positional aid device 500). It will be understood that the flexible layer structure 522 may have other thickness, size, or shape configurations than those described above, include those described with regard to additional embodiments herein.

In the illustrated embodiments, the flexible layer structure 522 of the positional aid device 500 defines a plurality of thru-passages 548. Thru-passages 548 are arranged in an array and generally incorporate the features described above with respect to thru-passages 48. Thru-passages 548 include identifying portion 510, first concave portion 512, and second concave portion 514. First concave portion 512 defines a concavity of decreasing dimension facing towards a first side 524 of patterned grid structure 500. Second concave portion 514 defines a concavity of decreasing dimension facing towards a second side 526 of patterned grid structure 500. Identifying portion 510 defines a concavity of consistent dimension in a plane perpendicular to the axis of the respective thru-passage 548. In this way, each thru-passage 548 includes a first rim 550 at first side 524 defined by identifying portion 510, and a second rim 552 at second side 526 defined by second concave portion 512. Identifying portion 510 extends through the thickness of the positional aid device at least a first distance 568 that represents only a portion of the thickness of the positional aid device. In some forms, distance 568 represents at least about 10% of the thickness of the positional aid device 500, and typically represents in the range of about 25% to about 75% of the thickness of the positional aid device 500. Additionally, or alternatively, distance 568 can be at least about 0.05 cm, or can be in the range of about 0.05 cm to about 1.5 cm.

As shown in the embodiments illustrated at FIGS. 26-29b and FIG. 31, identifying portions 510 are configured to define a variety of shapes to aid in visualization and location of a specific thru-passage, while maintaining the beneficial dual-concavity structure of the thru-passage allowing for improved interaction with a needle device at the first concavity and increased opening angles and needle guidance at the second concavity. In general, identifying portions comprise a geometric shape positioned around an opening of the thru-passage, preferably on at least the first side of the device. The identifying portion may comprise at least one surface defining the geometric shape and extending transverse to the axis of the thru-passage. In accordance with certain preferred embodiments, the identifying portions are configured such that the geometric shape is identifiable by MRI visualization around the thru-portion. Identifying portion 510 can be shaped to define any suitable shape identifiable by MRI, for example: circle, oval, or a polygon such as a square, triangle, parallelogram, trapezoid, pentagon, hexagon and/or diamond. In accordance with some forms a positional aid device as described herein may include a patterned grid structure having at least two unique shaped identifying portions, preferably at least three unique shaped identifying portions, more preferably at least four unique shaped identifying portions. In some forms a positional aid device of the present disclosure may include a patterned grid structure wherein each of the thru-portions has a uniquely shaped identifying portion, such that the device includes at least two identifying portions, each having an identifying portion shape different from the other. For example, in some forms, a device is provided comprising a first plurality of thru-passages having an identifying portion of a first shape, and a second plurality of thru-passages having identifying portions of a second shape, wherein the second shape differs from the first shape. In accordance with some forms, thru-portions having similarly shaped identifying portions may be grouped together to aid in localization. For example, the grid may be arranged in a quadrant system, as shown in FIGS. 26-28, and 31. In the illustrated embodiment, a first quadrant 570 comprises a plurality of thru-openings having circular shaped identifying portion. A second quadrant 572 comprises a plurality of thru-openings having square shaped identifying portions. A third quadrant 574 comprises a plurality of thru-openings having triangular shaped identifying portions. A fourth quadrant 576 comprises a plurality of thru-openings having diamond shaped identifying portions. Such a configuration may facilitate the visualization and division of each quadrant when looking at the grid on the patient, particularly when the device is only partially visible to a user. In some forms the device may comprise one or more quadrant barrier line 580 which provides additional visual location guidance. In some forms, the quadrant barrier line 580 is also MRI visible.

In accordance with some forms, positional aid device 500 comprises a flexible layer structure 522 and a plurality of rigid members positioned therein. One embodiment of such a device is illustrated in FIGS. 26, 27 and 29a. The flexible layer structure 522 includes a plurality of thru-passages 548, including identifying portions 510 on a first side 524 of flexible layer structure 522 as described above. In the illustrated embodiment, each of thru-passages 548 is configured to receive a rigid member 600, such that the through passage is defined by the identifying portion 510 on a first end toward the first surface 528, and the rigid member 600 on a second end towards the second surface 530. Rigid member 600 may be formed from a material that is more rigid than that of the flexible layer structure 522. The rigid members 600 in the illustrated embodiment each provide a portion of a respective thru-passage 548 and provide protective covers to a portion of the thru-passage. In the illustrated embodiment, the rigid members 600 provide a least the first concave portion 512, and second concave portion 514. The rigid member 600 can be attached to and contained within corresponding thru-passages. Such attachment may be achieved by any suitable means including as examples in-molding the rigid members 600 while molding the flexible layer structure 522 around them, by resiliently receiving and friction fitting the rigid members 600 within passages of the flexible layer structure 522, and/or by use of an adhesive.

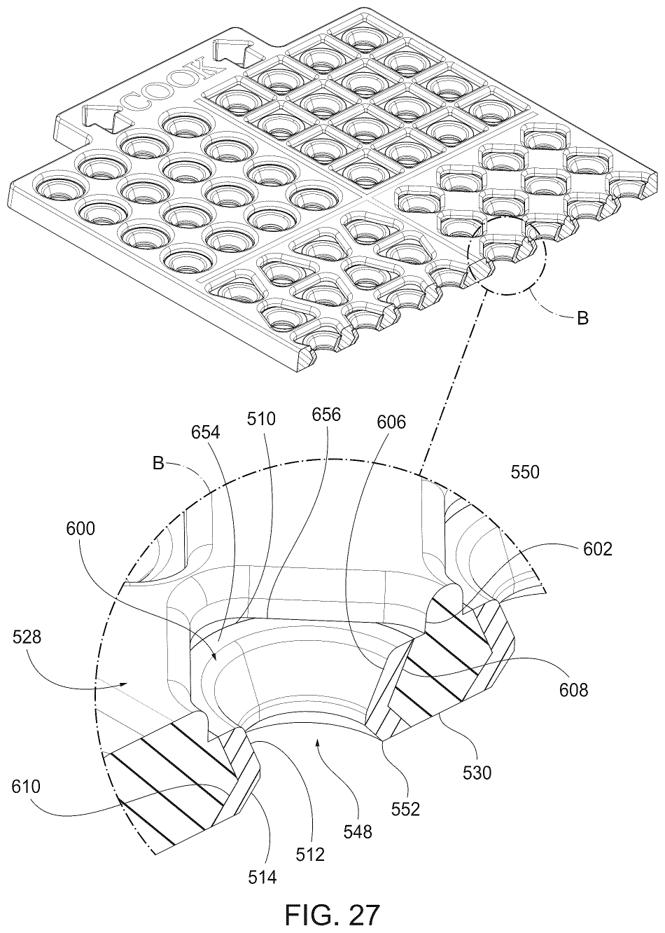

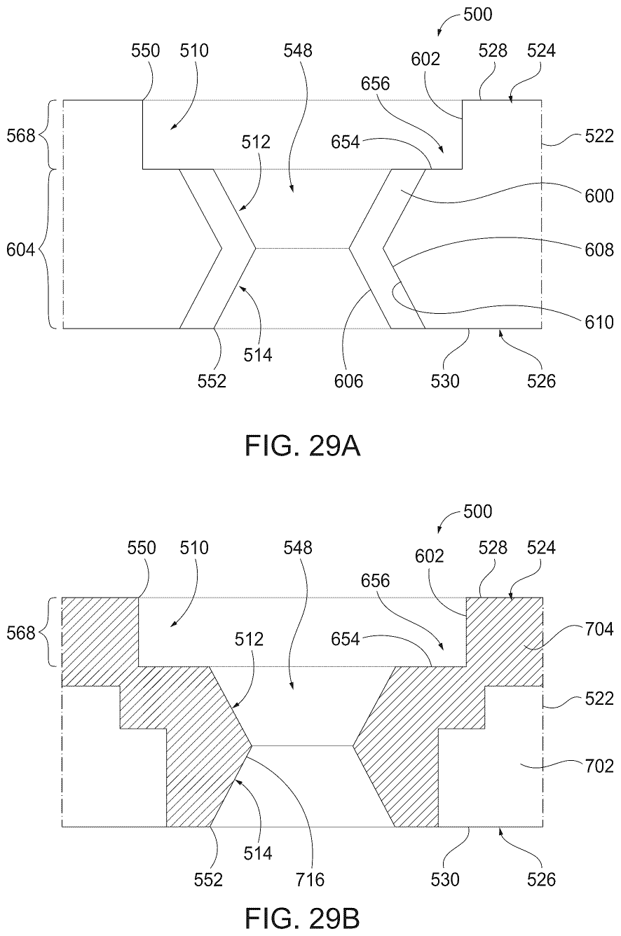

With reference to FIG. 29a, shown is a cross-sectional view of a single thru-passage 548 of a positional aid device 500 as described herein. FIG. 27 shows a cutaway view of a positional aid device 500 as shown in FIG. 26, along line A. FIG. 27 includes expanded portion B. Thru-passage 548 extends from a first side 524 of flexible layer structure 522, to a second side 526 of flexible layer structure 522. Thru-passage 548 are defined by identifying portion wall 602, and lumen wall 606 of rigid member 602. Outer wall 608 of rigid member 600 contacts opening wall 610 of flexible layer structure 522. identifying portion 510 extends into flexible body member a first distance 568. Rigid member 600 extends into flexible layer structure a second distance 604. In some forms the sum of distance 568 and distance 604 is equal to the total thickness of flexible layer structure 522. In certain embodiments, identifying portion comprises a shoulder portion 656 comprising wall 654, wherein shoulder wall 654 is roughly parallel to first surface 624, when the device is placed on a flat surface or otherwise in a planar condition.

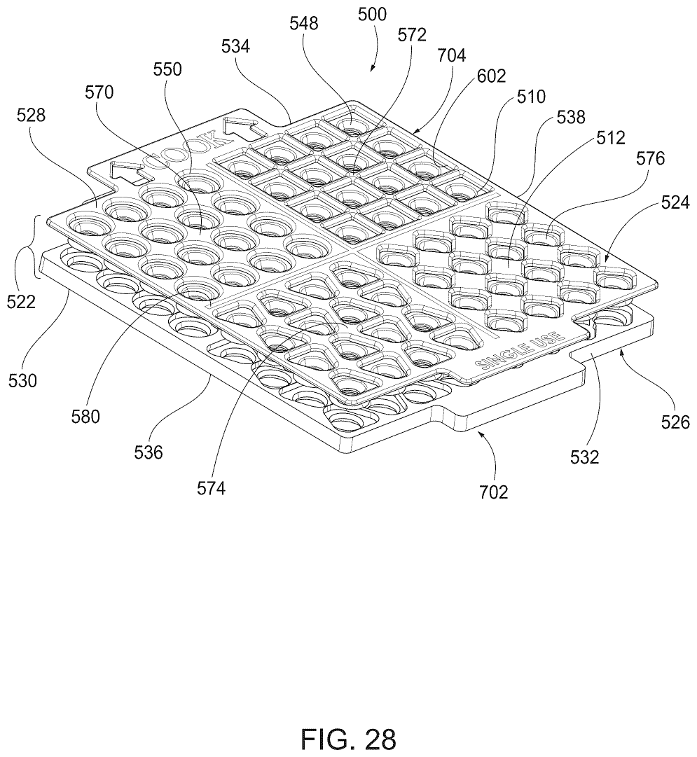

In accordance with some forms, the positional aid device 500 comprises a flexible layer structure 522 comprising a bilayer configuration having a base layer and a top layer. One embodiment of such a device is illustrated at FIGS. 28 and 29b. Except as detailed below, positional aid device 500 incorporates many of the features described above. In accordance with some forms, the base layer comprises a relatively more flexible material, for example having a lower durometer, than the material of the top layer. One or both of the layers may comprise an elastomeric polymeric material as disclosed herein, preferably an MRI imageable material. In the illustrated embodiment, positional aid device 500 comprises base layer 702 and top layer 704. Base layer 702 comprises a plurality of openings in a grid pattern corresponding to thru-passages 548 of top layer 704. In this way, the relatively rigid top layer provides the inner surface of the entirety of thru-passage 548 extending through the thickness of device 500.

With reference to FIG. 29b, shown is a cross-sectional view of a single thru-passage 548 of a positional aid device 500 according to one embodiment as described herein. Thru-passage 548 extends from a first side 524 of positional aid device 500, to a second side 526 of positional aid device 500. Thru-passage 548 is defined by identifying portion wall 602, lumen wall 714 of first concave portion 512, and lumen wall 716 of second concave portion 514. Identifying portion 510 extends into top layer 704 a first distance 568. In certain embodiments, identifying portion comprises a shoulder portion 656 comprising shoulder wall 654, wherein wall 654 is roughly parallel to first surface 528, when the device is placed on a flat surface.

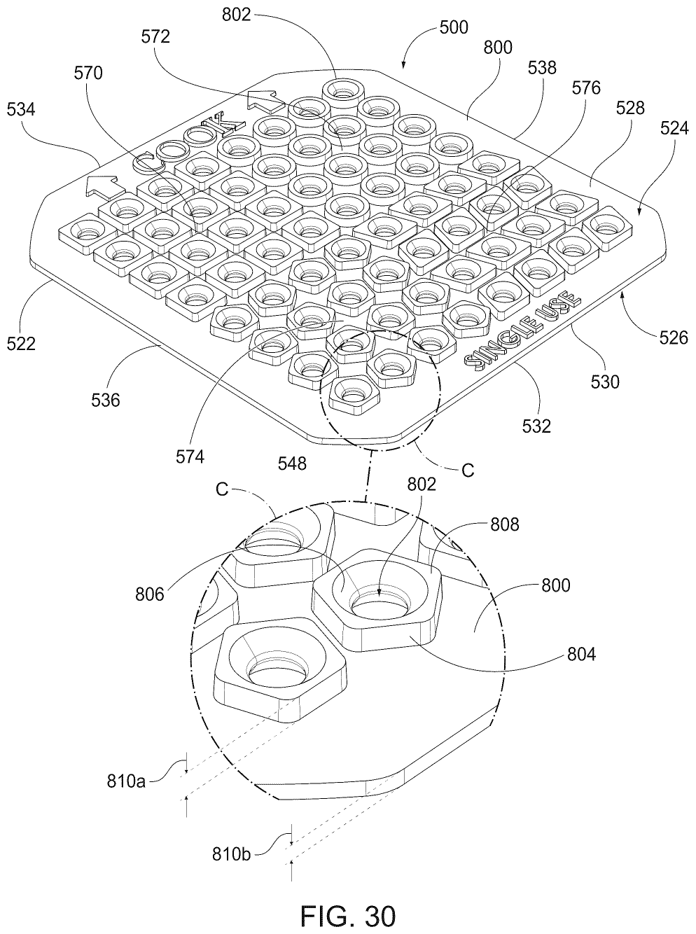

Turning now to the embodiment illustrated by FIG. 30, in accordance with some forms a positional aid device may comprise a flexible layer structure having raised, identifying portions. Such devices may be made, for example case or molded, from a single material such as an elastomeric polymeric material as described herein, preferably an elastic polymeric material that is visible under MRI. In accordance with some forms, such devices may comprise a flexible layer structure 522 comprising a base layer 800, having a plurality of raised identifying portions 802 positioned thereon. Flexible layer structure 522 comprises a first side 524 defining a first surface 528 and a second side 526 defining a second surface 530. Second surface 530 may be configured for comfortable contact and/or adhesion to patient skin as detailed above. In the illustrated embodiment, first side 524 comprises a plurality of raised identifying portions 802. Each of the raised identifying portions comprise an outer wall 804, a top surface 808, and an inner wall 806. Inner wall 806 defines thru-passage 548. Thu-passages 548 may include many of the features described above, for example having a first concave portion and/or a second concave portion. In certain preferred forms, outer walls 804 of adjacent raised identifying portions 802 are not connected to one another, except by the base layer. Such a configuration allows for increased flexibility of the device. As shown in the illustrated embodiment, raised identifying portions 804 may be configured such that top surface 808 and outer wall 804 form a shape useful for identifying a particular thru-passage. Such shapes may be any suitable shape as described herein, for example: a circle, oval, or a polygon such as a square, triangle, parallelogram, trapezoid, pentagon, hexagon and/or diamond.

Positional aid devices having raised identification portions may employ a grid structure as described above. For example, the grid may be arranged in a quadrant system, as shown in FIG. 30. In the illustrated embodiment, a first quadrant 570 comprises a plurality of raised identifying portions having a square shaped profile. A second quadrant 572 comprises a plurality of raised identifying portions having a circular shaped profile. A third quadrant 574 comprises a plurality of raised identifying portions having a pentagon shaped profile. A fourth quadrant 576 comprises a plurality of raised identifying portions having a parallelogram shaped profile. Such a configuration may facilitate the visualization and division of each quadrant when looking at the grid on the patient, particularly when the device is only partially visible to a user. When possible, shapes within a given profile may be positioned to distinguish one row from another. For example, as shown in the fourth quadrant 576 of FIG. 30 the parallelogram shaped raised identifying portions are aligned in rows having alternating angled sides such that each row is distinguishable from the adjacent rows.

Raised identifying portion 802 may have a height 810a, representing the distance from the first surface 528 to top surface 808. In some forms, the height 810a may be about 1 mm to about 8 mm, preferably about 1.5 mm to about 4 mm, more preferably about 2 mm to about 3 mm. In some forms, the height 810a is about 2 mm to about 2.5 mm. In some forms, height 810a is about 2.25 mm. Base layer 800 may have a thickness 810b, representing the distance between the first surface 528 and the second surface 530. In some forms, the thickness 810b may be about 0.5 mm to about 8 mm, preferably about 0.75 mm to about 4 mm, more preferably about 1 mm to about 2 mm. In some forms, the thickness 810b is about 1.25 mm to about 1.75 mm. In some forms, thickness 810a is about 1.5 mm. In this way, flexible layer structure 522 may have a maximum thickness comprising the sum of raised identifying portion height 810a and base layer thickness 810b. In accordance with some forms, the maximum thickness of flexible layer structure 522 is about 1 mm to about 20 mm, preferably about 1.5 mm to about 10 mm, more preferably about 2 mm to about 5 mm. In some forms, the maximum thickness of flexible layer structure 522 is about 3 mm to about 4 mm. In some forms, the maximum thickness of flexible layer structure 522 is about 3.75 mm.

In certain embodiments, the shaped profiles of each raised identifying portion are configured for MRI visibility. For example, the top surface may have a minimum surface thickness, comprising the minimum distance between a first rim, between top surface 808 and inner wall 806, to first edge, between top surface 808 and outer wall 804. In some forms the minimum surface thickness of top surface 808 is about 0.25 mm to about 3 mm. In other embodiments the top surface 808 has a top surface area of at least.

It is understood that the positional aid devices disclosed above comprising one or more identifying portions, may be used with an MRI imaging coil device as disclosed herein. Such MRI imaging coil devices may also include at least one mounting frame as detailed above.

It is intended that the foregoing detailed description be regarded as illustrative rather than limiting, and that it be understood that it is the following claims, including all equivalents, that are intended to define the spirit and scope of this disclosure. All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present disclosure, and is not intended to limit the present disclosure in any way to such theory, mechanism of operation, proof, or finding. As well, all steps of a given disclosed process or other combination of steps disclosed herein may be conducted in any suitable order unless otherwise expressly indicated, and thus the disclosure contemplates the order of steps as indicated herein, as well as other orders; and, for any process or other combination of steps described herein, it will be understood that not all of the disclosed steps are required in all embodiments herein unless otherwise expressly indicated.

While the disclosure has been illustrated and described in detail in the drawings and foregoing description, the same is to be considered as illustrative and not restrictive in character, it being understood that only selected embodiments have been shown and described and that all equivalents, changes, and modifications that come within the spirit of the disclosures as defined herein or by the following claims are desired to be protected.

### Glossary of Terms

The language used in the claims and specification is to only have its plain and ordinary meaning, except as explicitly defined below. The words in these definitions are to only have their plain and ordinary meaning. Such plain and ordinary meaning is inclusive of all consistent dictionary definitions from the most recently published Webster's dictionaries and Random House dictionaries. As used in the specification and claims, the following definitions apply to these terms and common variations thereof identified below.

“MRI visible material” generally means any material capable of emitting a signal imageable by an MRI machine. Suitable MRI visible materials include aqueous media containing paramagnetic materials such as nickel, manganese, copper sulfate, sodium chloride, gadolinium chloride, and/or an oil. In certain embodiments, the MRI visible material comprises a solid or semi-solid medium containing polyvinyl alcohol, wax, a superabsorbent polymer (e.g. a polyacrylic acid superabsorbent polymer), gelatin, and/or agarose. In certain embodiments an MRI visible material may contain a salt, such as sodium chloride, nickel sulfate, and/or thulium nitrate, effective to modify its T1 or T2 relaxation time under magnetic resonance. In any of the embodiments described herein, the MRI visible material may comprise any liquid, gel, solid, and/or semi-solid material with response under MRI configured to provide contrast and thereby render visible all, a selected portion, and/or selected portions, of the guide device in the image generated by MRI, for example when positioned external of the patient in the ex vivo gaseous environment (e.g. air) surrounding the patient.

“Van der Waals interactions” generally means weak, non-covalent forces between atoms or molecules that play a crucial role in the structure and behavior of different substances. They arise from temporary fluctuations in electron distribution, leading to transient dipoles (London dispersion forces), and from permanent dipoles in polar molecules (dipole-dipole interactions). These interactions, though individually weak compared to covalent or ionic bonds, collectively influence phenomena like boiling points, viscosity, and the physical properties of materials, especially in organic compounds and biomolecules. In certain applications, the effect of these interactions is particularly pronounced in materials with large, easily polarizable molecules, resulting in adhesion even without the presence of covalent or ionic bonds.

“Needle shaft” generally refers to the long, slender part of a needle extending from the tip, which is the pointed end designed to penetrate skin or other materials, to the base, where it typically connects to a hub or handle. In medical contexts, the needle shaft is a critical component of hypodermic needles, biopsy needles, or other percutaneous intervention tools. The design of the shaft, including its length, diameter, and flexibility, varies depending on its intended use. For instance, longer shafts may be used for deeper penetrations into tissue, while thinner shafts can minimize patient discomfort. The surface of the shaft may also have specific features, like coatings of MRI-visible materials or textures, to improve performance and/or patient safety.

“Computer” generally refers to any computing device configured to compute a result from any number of input values or variables. A computer may include a processor for performing calculations to process input or output. A computer may include a memory for storing values to be processed by the processor, or for storing the results of previous processing. A computer may also be configured to accept input and output from a wide array of input and output devices for receiving or sending values. Such devices include other computers, keyboards, mice, visual displays, printers, industrial equipment, and systems or machinery of all types and sizes. For example, a computer can control a network interface to perform various network communications upon request. A computer may be a single, physical, computing device such as a desktop computer, a laptop computer, or may be composed of multiple devices of the same type such as a group of servers operating as one device in a networked cluster, or a heterogeneous combination of different computing devices operating as one computer and linked together by a communication network. A computer may include one or more physical processors or other computing devices or circuitry and may also include any suitable type of memory. A computer may also be a virtual computing platform having an unknown or fluctuating number of physical processors and memories or memory devices. A computer may thus be physically located in one geographical location or physically spread across several widely scattered locations with multiple processors linked together by a communication network to operate as a single computer. The concept of “computer” and “processor” within a computer or computing device also encompasses any such processor or computing device serving to make calculations or comparisons as part of a disclosed system. Processing operations related to threshold comparisons, rules comparisons, calculations, and the like occurring in a computer may occur, for example, on separate servers, the same server with separate processors, or on a virtual computing environment having an unknown number of physical processors as described above.

“Array” generally refers to a structured arrangement of items, often in a regular pattern. For example, an array may be a grid including a network of intersecting parallel lines, either horizontal and vertical or diagonal, creating a pattern of squares or rectangles, or an arrangement of concentric lines each defining a closed shape, for example an arrangement of concentric circular lines. An array in the form of a grid can be an orderly arrangement of similar objects in rows and columns.

“Edge” generally refers to a border where an object or area begins or ends. The edge is typically in the form of a line or line segment that is at the intersection of two plane faces or of two planes of an object or space.

“and/or” generally refers to a grammatical conjunction indicating that one or more of the cases it connects may occur. For instance, it can indicate that either or both of the two stated cases can occur. In general, “and/or” includes any combination of the listed collection. For example, “X, Y, and/or Z” encompasses: any one letter individually (e.g., {X}, {Y}, {Z}); any combination of two of the letters (e.g., {X, Y}, {X, Z}, {Y, Z}); and all three letters (e.g., {X, Y, Z}). Such combinations may include other unlisted elements as well.

“Ex vivo” generally refers to processes and/or experiments conducted outside of a living organism. This term, which literally means “out of the living” in Latin, is distinct from “in vivo” (within the living) and “in vitro” (in the laboratory).

It should be noted that the singular forms “a,” “an,” “the,” and the like as used in the description and/or the claims include the plural forms unless expressly discussed otherwise. For example, if the specification and/or claims refer to “a device” or “the device”, it includes one or more of such devices.

It should be noted that directional terms, such as “up,” “down,” “top,” “bottom,” “lateral,” “longitudinal,” “radial,” “circumferential,” “horizontal,” “vertical,” etc., are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the disclosures defined by the following claims are desired to be protected. All publications, patents, and patent applications cited in this specification are herein incorporated by reference as if each individual publication, patent, or patent application were specifically and individually indicated to be incorporated by reference and set forth in its entirety herein.

### Embodiments

1. A magnetic resonance imaging (MRI) positional aid device, comprising:

a flexible layer structure having a first side and a second side opposite the first side; and

the flexible layer structure defining a plurality of thru-passages, wherein the thru-passages extend completely through a thickness of the flexible layer structure, and wherein the thru-passages are demarked by an MRI visible material of the flexible layer structure.

2. The device of embodiment 1, wherein the plurality of thru-passages are in a defined array and/or wherein each thru-passage of the plurality of thru-passages forms a concavity of decreasing dimension as it extends from the first side toward the second side.
3. The device of embodiment 1 or 2, wherein the flexible layer structure includes interior passages and the MRI visible material is contained in the interior passages; and optionally wherein the MRI visible material includes water.
4. The device of embodiment 1 or 2, wherein the flexible layer structure includes a polymeric material and the MRI visible material is dispersed within the polymeric material.
5. The device of any preceding embodiment, wherein the flexible layer structure has a thickness of less than about 2 cm, less than about 1 cm, at least about 0.5 cm, or in the range of about 0.5 cm to about 2 cm.
6. The device of any preceding embodiment, wherein the thru-passages have a non-constant diameter.
7. The device of any preceding embodiment, wherein the array is a grid array having a first axis and a second axis perpendicular to the first axis.
8. The device of any preceding embodiment, also comprising an adhesive on the second side configured to temporarily maintain a fixed position of the device on patient tissue.
9. The device of any preceding embodiment, wherein the flexible layer structure also includes visible indicia associated with the array, wherein the visible indicia include MRI-visible indicia and/or directly visible indicia.
10. The device of embodiment 9, wherein a first set of the visible indicia is correlated to a first axis along the array and a second set of the visible indicia is correlated to a second axis along the array.
11. The device of embodiment 9, wherein the visible indicia include device identification indicia.
12. The device of any one of embodiments 9 to 11, wherein the visible indicia include MRI-visible indicia.
13. The device of embodiment 12, wherein the MRI-visible indicia include a first set of MRI-visible indicia correlated to a first axis along the array and a second set of MRI-visible indicia correlated to a second axis along the array, the second axis being perpendicular to the first axis.
14. The device of embodiment 13, wherein the MRI-visible indicia also include a device identification indicia.
15. The device of any one of embodiments 1 to 14, wherein the MRI visible material is contained within one or more internal cavities defined in the flexible layer structure.
16. The device of embodiment 15, wherein the one or more internal cavities form a fluidly continuous internal passageway system containing amounts of the MRI visible material demarking at least first and second thru-passages of the plurality of thru-passages, preferably wherein the fluidly continuous internal passageway system contains amounts of the MRI visible material demarking all of the plurality of thru-passages.
17. The device of embodiment 15 or 16, wherein the MRI visible material is a three-dimensionally stable solid.
18. The device of any one of embodiments 1 to 17, wherein the flexible layer structure also includes a first plurality of protective covers positioned at the first side, the protective covers made of a rigid polymeric material and each defining at least a portion of a respective thru-passage of the plurality of thru-passages.
19. The device of embodiment 18, wherein the protective covers have walls that define concavities of diminishing dimension in a direction from the first side to the second side of the flexible layer structure.
20. The device of embodiment 18 or 19, wherein the flexible layer structure further includes a second plurality of protective covers positioned at the second side, the protective covers of the second plurality of protective covers made of a rigid polymeric material and each defining at least a portion of a respective thru-passage of the plurality of thru-passages.
21. The device of embodiment 20, wherein the protective covers of the second plurality of protective covers have walls that define concavities of diminishing dimension in a direction from the second side to the first side of the flexible layer structure.
22. The device of any one of embodiments 17 to 21, wherein the protective covers comprise the MRI visible material
23. The device of any one of embodiments 1 to 22, wherein the MRI visible material also defines at least one asymmetric feature.
24. The device of any one of embodiments 1 to 23, wherein the flexible layer structure is comprised of an elastomeric polymeric material.
25. The device of any one of embodiments 1 to 24, wherein the thru-passages of the plurality of thru-passages constitute all thru-passages that extend completely through the thickness of the flexible layer structure.
26. The device of any one of embodiments 1 to 25, wherein each thru-passage of the plurality of thru-passages forms a first concavity of decreasing dimension as it extends from the first side toward the second side.
27. The device of embodiment 26, wherein the first concavity is bowl-shaped or cone-shaped.
28. The device of embodiment 26 or 27, wherein the first concavity defines a continuously curved cross-sectional shape in a plane perpendicular to the axis of the thru-passage defining the first concavity; optionally wherein the cross-sectional shape is a circular cross-sectional shape.
29. The device of any one of embodiments 26 to 28, wherein the first concavity is defined by walls that extend at an angle relative to a surface of the first side and through the thickness of the flexible layer structure a distance that (i) represents only a portion of the thickness and/or (ii) is least about 0.05 cm, or in the range of about 0.05 cm to about 1.5 cm.
30. The device of embodiment 29, wherein said angle is in the range of about 20 to about 70 degrees, or in the range of about 30 to about 55 degrees.
31. The device of embodiment 29 or 30, wherein said distance is at least about 10% of the thickness of the flexible layer structure, or is in the range of about 25% to about 75% of the thickness of the layer structure.
32. The device of any one of embodiments 1 to 31, wherein each thru-passage of the plurality of thru-passages forms a second concavity of decreasing dimension as it extends from the second side toward the first side.
33. The device of embodiment 32, wherein the second concavity is bowl-shaped or cone-shaped.
34. The device of embodiment 32 or 33, wherein the second concavity defines a continuously curved cross-sectional shape in a plane perpendicular to an axis of the thru-passage defining the second concavity; optionally wherein the cross-sectional shape is a circular cross-sectional shape.
35. The device of any one of embodiments 32 to 34, wherein the second concavity is defined by walls that extend at a second angle relative to a surface of the second side and through the thickness of the flexible layer structure a second distance that is least about 0.05 cm, or in the range of about 0.05 cm to about 1.5 cm.
36. The device of embodiment 35, wherein said second angle is in the range of about 20 to about 70 degrees, or in the range of about 30 to about 55 degrees.
37. The device of embodiment 35 or 36, wherein said second distance is at least about 10% of the thickness of the flexible layer structure.
38. The device of any one of embodiments 1 to 37, wherein the flexible layer structure comprises a natural or synthetic rubber.
39. The device of any one of embodiments 1 to 38, sterilely received in a moisture-proof package, optionally a foil package.
40. A method for using a positional aid device, comprising:

providing a positional aid device according to any one of embodiments 1 to 38, or claims 160 to 178; and

performing magnetic resonance (MR) imaging of a target imaging region including the device, the MR imaging generating and displaying at least one MR image in which said thru-passages are demarked in the MR image by the MRI visible material.

41. The method of embodiment 40, further comprising:

engaging an interventional device with a contact surface of the flexible layer structure that is adjacent to one of the plurality of thru-passages.

42. The method of embodiment 41, wherein the engaging includes contacting a contact surface of the interventional device with the contact surface of the flexible layer structure; optionally wherein the contact surface of the interventional device is contoured to conform to the contact surface of the flexible layer structure.
43. The method of embodiment 42, wherein the contact surface of the interventional device and the contact surface of the flexible layer cooperate to provide an advancement stop to the interventional device such that the interventional device penetrates only partially into the thickness of the positional aid device during said engaging.
44. The method of any one of embodiments 41 to 43, wherein the interventional device comprises an MRI visible material, and wherein the target imaging region includes the interventional device and the at least one MR image includes the interventional device engaged with the contact surface of the flexible layer structure.
45. The method of embodiment 44, wherein the MRI visible material of the interventional device demarks an elongate longitudinal path along a length of the interventional device.
46. The method of embodiment 44 or 45, wherein the interventional device includes a guide device configured to guide an insertion path of a tissue penetrating device.
47. The method of embodiment 45, wherein the interventional device includes a guide device configured to guide an insertion path of a tissue penetrating device, wherein the MRI visible material is in the guide device, and wherein said insertion path extends along the elongate longitudinal path demarked by the MRI visible material.
48. The method of embodiment 47, wherein the MRI visible material includes a first material portion laterally adjacent the insertion path to a first lateral side thereof and a second material portion laterally adjacent the insertion path to a second lateral side thereof opposite the first lateral side.
49. The method of any one of embodiments 46 to 48, wherein the tissue penetrating device comprises a needle.
50. The method of embodiment 49, wherein the guide device defines a passageway along the insertion path for receiving and guiding movement of a needle shaft of the needle.
51. The method of embodiment 50, wherein the passageway is a groove; optionally wherein the groove is a longitudinal groove along a side of the instrument configured to laterally receive the tissue penetrating device.
52. The method of any one of embodiments 40 to 51, wherein during said performing MR imaging the positional aid device is received adjacent to tissue of a patient, and optionally against the tissue of the patient.
53. The method of embodiment 52, wherein the tissue is skin tissue.
54. The method of any one of embodiments 46 to 51, wherein during said performing MR imaging the positional aid device is received adjacent to tissue of a patient, the method further including moving a tissue penetrating device along the insertion path so as to insert the tissue penetrating device into the patient.
55. The method of embodiment 54, which is a biopsy method, the method also including obtaining a tissue sample from an internal region in the patient with the tissue penetrating device.
56. The method of embodiment 55, wherein the target imaging region also includes the internal region of the patient and the at least one MR image also includes the internal region of the patient.
57. The method of any one of embodiments 40 to 56, wherein said MR imaging is real-time MR imaging and said generating and displaying at least one MR image includes generating and displaying real-time MR images.
58. The method of any one of embodiments 42 to 57 as dependent on claim 41, wherein said engaging comprises orienting the interventional device relative to the positional aid device manually by hand.
59. The method of embodiment 58, wherein said orienting is conducted during and/or following visually observing the at least one MR image; optionally wherein the at least one MR image visibly identifies a target pathway and wherein said orienting is at least in part based on visually observing the identified target pathway.
60. The method of embodiment 59, wherein the generating and displaying at least one MRI image includes generating and simultaneously displaying a first two-dimensional MRI image and a second two-dimensional MRI image slice intersecting the first two-dimensional MRI image, and wherein the first and second two-dimensional MRI images together identify the target pathway.
61. A positional aid device for use with a medical imaging system, comprising:

a layer structure having a first side and a second side opposite the first side; and

the layer structure defining a plurality of thru-passages;

wherein the thru-passages extend completely through a thickness of the layer structure;

wherein the thru-passages are demarked by a visible material of the layer structure visible in images generated by the medical imaging system; and

wherein the thru-passages of the plurality of thru-passages each form a first concavity at the first side of decreasing dimension as it extends from the first side toward the second side.

62. The device of embodiment 61, wherein the layer structure includes one or more internal cavities and the material visible in images generated by the medical imaging system is contained in the one or more cavities.
63. The device of embodiment 60 or 61, wherein the thru-passages of the plurality of thru-passages are in a defined array.
64. The device of embodiment 63, wherein the layer structure also includes visible indicia associated with the array, wherein the visible indicia include directly visible indicia and/or indicia visible in images generated by the medical imaging system; optionally wherein a first set of the visible indicia is correlated to a first axis of the array and a second set of visible indicia is correlated to a second axis of the array.
65. The device of embodiment 64, wherein the layer structure also includes device identification visible indicia.
66. The device of any one of embodiments 61 to 65, wherein the medical imaging system is a magnetic resonance imaging system or an X-ray imaging system.
67. The device of any one of embodiment 61 to 66, wherein the layer structure has a thickness of at least about 0.5 cm, or in the range of about 0.5 cm to about 2 cm; and/or wherein the thru-passages of the plurality of thru-passages each form a second concavity of decreasing dimension as it extends from the second side toward the first side.
68. The device of embodiment 67, wherein the thru-passages of the plurality of thru-passages each have a smallest cross-sectional dimension occurring between the first side and the second side of the layer structure.
69. The device of any one of embodiments 61 to 68, wherein the layer structure includes interior passages and the visible material is contained in the interior passages; and optionally wherein the visible material includes water.
70. The device of any one of embodiments 61 to 68, wherein the layer structure includes a polymeric material and the visible material is dispersed within the polymeric material.
71. The device of any one of embodiments 61 to 70, wherein the layer structure has a thickness of less than about 2 cm, less than about 1 cm, at least about 0.5 cm, or in the range of about 0.5 cm to about 2 cm.
72. The device of any one of embodiments 61 to 71, wherein the thru-passages of the plurality of thru-passages are arranged in a grid array having a first axis and a second axis perpendicular to the first axis.
73. The device of any one of embodiments 61 to 72, also comprising an adhesive on the second side configured to temporarily maintain a fixed position of the device on patient tissue.
74. The device of any one of embodiments 61 to 73, wherein the visible material is contained within one or more internal cavities defined in the layer structure.
75. The device of embodiment 74, wherein the one or more internal cavities form a fluidly continuous internal passageway system containing amounts of the visible material demarking at least first and second thru-passages of the plurality of thru-passages, preferably wherein the fluidly continuous internal passageway system contains amounts of the visible material demarking all of the plurality of thru-passages.
76. The device of embodiment 74 or 75, wherein the MRI visible material is a three-dimensionally stable solid.
77. The device of any one of embodiments 61 to 76, wherein the layer structure also includes a first plurality of protective covers positioned at the first side, the protective covers made of a rigid polymeric material and each defining at least a portion of a respective thru-passage of the plurality of thru-passages.
78. The device of embodiment 77, wherein the protective covers have walls that define concavities of diminishing dimension in a direction from the first side to the second side of the layer structure.
79. The device of embodiment 77 or 78, wherein the layer structure further includes a second plurality of protective covers positioned at the second side, the protective covers of the second plurality of protective covers made of a rigid polymeric material and each defining at least a portion of a respective thru-passage of the plurality of thru-passages.
80. The device of embodiment 79, wherein the protective covers of the second plurality of protective covers have walls that define concavities of diminishing dimension in a direction from the second side to the first side of the layer structure.
81. The device of any one of embodiments 77 to 80, wherein the protective covers comprise the MRI visible material
82. The device of any one of embodiments 61 to 81, wherein the visible material also defines at least one asymmetric feature.
83. The device of any one of embodiments 61 to 82, wherein the layer structure is comprised of an elastomeric polymeric material.
84. The device of any one of embodiments 61 to 83, wherein the thru-passages of the plurality of thru-passages constitute all thru-passages that extend completely through the thickness of the layer structure.
85. The device of any one of embodiments 61 to 84, wherein the first concavity is bowl-shaped or cone-shaped.
86. The device of any one of embodiments 61 to 85, wherein the first concavity defines a continuously curved cross-sectional shape in a plane perpendicular to the axis of the thru-passage defining the first concavity; optionally wherein the cross-sectional shape is a circular cross-sectional shape.
87. The device of any one of embodiments 61 to 86, wherein the first concavity is defined by walls that extend at an angle relative to a surface of the first side and through the thickness of the layer structure a distance that (i) represents only a portion of the thickness and/or (ii) is least about 0.05 cm, or in the range of about 0.05 cm to about 1.5 cm.
88. The device of embodiment 87, wherein said angle is in the range of about 20 to about 70 degrees, or in the range of about 30 to about 55 degrees.
89. The device of embodiment 87 or 88, wherein said distance is at least about 10% of the thickness of the layer structure, or is in the range of about 25% to about 75% of the thickness of the layer structure.
90. The device of any one of embodiments 61 to 89, wherein each thru-passage of the plurality of thru-passages forms a second concavity of decreasing dimension as it extends from the second side toward the first side.
91. The device of embodiment 90, wherein the second concavity is bowl-shaped or cone-shaped.
92. The device of embodiment 90 or 91, wherein the second concavity defines a continuously curved cross-sectional shape in a plane perpendicular to an axis of the thru-passage defining the second concavity; optionally wherein the cross-sectional shape is a circular cross-sectional shape.
93. The device of any one of embodiments 90 to 92, wherein the second concavity is defined by walls that extend at a second angle relative to a surface of the second side and through the thickness of the layer structure a second distance that is least about 0.05 cm, or in the range of about 0.05 cm to about 1.5 cm.
94. The device of embodiment 93, wherein said second angle is in the range of about 20 to about 70 degrees, or in the range of about 30 to about 55 degrees.
95. The device of embodiment 93 or 94, wherein said second distance is at least about 10% of the thickness of the layer structure.
96. The device of any one of embodiments 61 to 95, wherein the layer structure comprises a natural or synthetic rubber and/or is a flexible layer structure.
97. The device of any one of embodiments 61 to 96, sterilely received in a medical package.
98. The device of embodiment 97, wherein the medical package is a moisture-proof package, optionally a foil package.
99. A procedural system, comprising:
(a) a positional aid device comprising:
   a layer structure having a first side and a second side opposite the first side;
   the layer structure defining a plurality of thru-passages;
   wherein the thru-passages extend completely through a thickness of the layer structure; and
   wherein the thru-passages are demarked by a visible material of the layer structure visible in images generated by the medical imaging system; and
(b) a guide device for guiding an interventional instrument, the guide device comprising a visible material visible in images generated by the medical imaging system.
   100. The system of embodiment 99, wherein the positional aid device is a device according to any one of embodiments 61 to 98.
   101. The system of embodiment 99 or 100, wherein the visible material of the guide device demarks an elongate longitudinal path along a length of the guide device.
   102. The system of any one of embodiments 99 to 101, wherein the interventional instrument is a tissue penetrating device, and wherein the guide device is configured to guide the tissue penetrating device along an insertion path.
   103. The system of embodiment 102, wherein said insertion path extends along the elongate longitudinal path demarked by the visible material of the guide device.
   104. The system of embodiment 103, wherein the visible material of the guide device includes a first visible material portion laterally adjacent the insertion path to a first lateral side thereof and a second visible material portion laterally adjacent the insertion path to a second lateral side thereof opposite the first lateral side.
   105. The system of any one of embodiments 102 to 104, wherein the tissue penetrating device comprises a needle.
   106. The system of any one of embodiments 102 to 105, wherein the guide device defines a passageway along the insertion path for receiving and guiding movement of a shaft of the tissue penetrating device.
   107. The system of embodiment 106, wherein the passageway is a groove.
   108. The system of embodiment 107, wherein the groove is a longitudinal groove along a side of the guide device configured to laterally receive the shaft of the tissue penetrating device.
   109. The system of any one of embodiments 99 to 108, also comprising the interventional instrument.
   110. The system of embodiment 109, wherein the interventional instrument is a biopsy device.
   111. The system of any one of embodiments 99 to 110, wherein the guide device has a distal tip contoured to conform with respective surface portions of the first side surrounding the thru-passages of the plurality of thru-passages; optionally wherein the distal tip is a convexly rounded distal tip.
   112. A method of using a procedural system, comprising:

providing a system according to any one of embodiments 99 to 111; and

engaging the guide device with a surface portion on the first side surrounding a selected one of the plurality of thru-passages.

113. The method of embodiment 112, also comprising generating an image with the medical imaging system while the guide device is engaged with the surface portion on the first side.
114. The method of embodiment 113, wherein the medical imaging system is a magnetic resonance imaging system or an X-ray imaging system.
115. A medical kit, comprising:

a system according to any one of embodiments 99 to 111; and

a kit package containing the system.

116. An MR imaging coil device configured for use with a positional aid device, comprising:

a flexible base layer having a thru-opening;

a loop antenna positioned in the base layer, optionally around the thru-opening;

at least one mounting frame attached to the base layer and defining a frame opening aligned with the thru-opening of the base layer and configured to receive a positional aid device;

wherein the MRI imaging coil device, and preferably the at least one mounting frame thereof, includes at least one fixation actuator movable between a first position configured to fix the positional aid device to the mounting frame while received in the frame opening and a second position configured to unfix the positional aid device from the mounting frame.

117. The MR imaging coil device of embodiment 116, wherein the fixation actuator includes at least one projecting member configured for contacting the positional aid device when the fixation actuator is moved from the first position to the second position; optionally wherein the at least one projecting member is configured for receipt within a fixation opening of the positional aid device when the fixation actuator is moved from the first position to the second position.
118. The MR imaging coil device of embodiment 116 or 117, wherein the fixation actuator is biased toward the first position, optionally spring biased toward the first position.
119. The MR imaging coil device of any one of embodiments 116 to 118, wherein the mounting frame has a peripheral edge defining a peripheral slot, and wherein portions of the flexible base layer are received within the peripheral slot.
120. The MR imaging coil device of any one of embodiments 116 to 119, wherein the frame opening has a first edge defined by a first frame wall of the mounting frame and a second edge opposite the first edge and defined by a second frame wall of the mounting frame, and wherein a first support member for supporting the positional aid device extends from and transversely to the first frame wall and into the frame opening, and wherein a second support member extends from and transversely to the second frame wall and into the frame opening.
121. The MR imaging coil device of embodiment 120, wherein the frame opening has a third edge defined by a third frame wall of the mounting frame, wherein the mounting frame further includes a third support member for supporting the positional aid device when received in the frame opening.
122. The MR imaging coil device of embodiment 121, wherein the third support member extends from and transversely to the third frame wall and into the frame opening.
123. The MR imaging coil device of embodiment 122, wherein the frame opening has a fourth edge opposite the third edge and defined by a fourth frame wall, and wherein the fixation actuator is positioned along the fourth frame wall.
124. The MR imaging coil device of any one of embodiments 116 to 123, also comprising the positional aid device, wherein the positional aid device is received within the frame opening.
125. The MR imaging coil device of embodiment 124, wherein the positional aid device is a device according to any one of embodiments 1 to 29 or 47 to 55, optionally received in a mount housing.
126. The MR imaging coil device of any one of embodiments 116 to 125, comprising a plurality of said mounting frames at spaced positions on the coil device.
127. The MR imaging coil device of any one of embodiments 116 to 126, wherein said at least one mounting frame is configured to receive the positional aid device in any one of multiple rotational orientations relative to the frame opening.
128. The MR imaging coil device of embodiment 127, wherein the fixation actuator is movable between the first position configured to fix the positional aid device to the mounting frame and the second position configured to unfix the positional aid device from the mounting frame with the positional aid device in each of the multiple rotational orientations.
129. The MR imaging coil device of any one of embodiments 116 to 128, wherein the at least one mounting frame includes the fixation actuator.
130. A method for MR imaging of a patient, comprising:

acquiring MR imaging data of a region including a positional aid device adjacent a patient with an MR imaging system; and

based at least in part on the MR imaging data, conducting at least one of, at least two of, at least three of, or all of, the following steps, using a computer processor:
(a) determining the presence of the positional aid device;
(b) determining one or more attributes of the positional aid device;
(c) determining an orientation of the positional aid device;
(d) determining a distance from a feature of the positional aid device to a tissue region of interest within the patient; and optionally displaying on an electronic display a numeric value representative of said distance from a feature;
(e) determining a target pathway from a feature of the positional aid device to a tissue region of interest within the patient, optionally wherein the feature is a thru-passage of the positional aid device selected from a plurality of thru-passages of the positional aid device; and optionally displaying on an electronic display a graphic representing the target pathway;
(f) determining a thru-passage of the positional aid device selected from a plurality of thru-passages of the positional aid device, acceptable for introducing an interventional instrument therethrough to access a tissue region of interest in the patient; and optionally displaying on an electronic display one or more alphanumeric characters and/or one or more graphics identifying the selected thru-passage;
(g) determining an instrument parameter for accessing a tissue region of interest within the patient through a thru-passage of the positional aid device;
(h) determining that a repositioning of the positional aid device relative to the patient is needed or recommended; and optionally notifying a user thereof visually on an electronic display and/or audibly through a speaker;
(i) determining that a repositioning of the patient is needed or recommended; and optionally notifying a user thereof visually on an electronic display and/or audibly through a speaker;
(j) determining one or more imaging sequence parameters to be used in the MR imaging of the patient; and optionally implementing the one or more imaging sequence parameters;
(k) determining that an interventional instrument to be used with the positional aid device has been engaged with a thru-passage of the positional aid device other than a thru-passage of the positional aid device that has been determined as acceptable for introduction of the interventional instrument therethrough to access a tissue region of interest in the patient; and optionally notifying a user thereof visually on an electronic display and/or audibly through a speaker;
(l) determining in real time a distance of an interventional instrument from a tissue region of interest as the interventional instrument extends through a thru-passage of the positional aid device and moves relative to the tissue region of interest; and optionally notifying a user thereof in real time visually on an electronic display and/or audibly through a speaker;
(m) determining in real time that interventional instrument moving through tissue of the patient has deviated significantly from a target pathway from a thru-passage of the positional aid device to a tissue region of interest in the patient; and optionally notifying a user thereof in real time visually on an electronic display and/or audibly through a speaker; or
(n) determining in real time that interventional instrument extending through a thru-passage of the positional aid device and moving through tissue of the patient has reached a tissue region of interest in the patient; and optionally notifying a user thereof in real time visually on an electronic display and/or audibly through a speaker.
   131. The method of embodiment 130, wherein said conducting comprises conducting at least step (f), and wherein said determining a thru-passage is based at least in part on determining a limited range of angles of an interventional instrument available when using the interventional instrument positioned through a thru-passage of the positional aid device.
   132. The method of embodiment 131, also comprising conducting at least step (b), and wherein said determining a limited range of angles occurs in step (b).
   133. The method of embodiment 131, wherein said conducting comprises conducting at least step (b), and wherein said one or attributes includes a limited range of angles of an interventional instrument available when using the interventional instrument positioned through a thru-passage of the positional aid device.
   134. The method of any one of embodiments 130 to 133, wherein the positional aid device is a device according to any one of embodiments 1 to 29, 47 to 55, or 160 to 178.
   135. A system for imaging of a patient, comprising:

a magnetic resonance imaging (MRI) system configured to acquire MRI data of a region including a positional aid device adjacent a patient; and

wherein the MRI system is configured to perform, based at least in part on the MRI data, at least one of, at least two of, at least three of, or all of, the following operations, using a computer processor:
(a) determining the presence of the positional aid device;
(b) determining one or more attributes of the positional aid device;
(c) determining an orientation of the positional aid device;
(d) determining a distance from a feature of the positional aid device to a tissue region of interest within the patient; and optionally displaying on an electronic display a numeric value representative of said distance from a feature
(e) determining a target pathway from a feature of the positional aid device to a tissue region of interest within the patient, optionally wherein the feature is a thru-passage of the positional aid device selected from a plurality of thru-passages of the positional aid device; and optionally displaying on an electronic display a graphic representing the target pathway;
(f) determining a thru-passage of the positional aid device selected from a plurality of thru-passages of the positional aid device, acceptable for the introduction of an interventional instrument therethrough to access a tissue region of interest in the patient; and optionally displaying on an electronic display one or more alphanumeric characters and/or one or more graphics identifying the selected thru-passage;
(g) determining an instrument parameter for accessing a tissue region of interest within the patient through a thru-passage of the positional aid device;
(h) determining that a repositioning of the positional aid device relative to the patient is needed or recommended; and optionally notifying a user thereof visually on an electronic display and/or audibly through a speaker;
(i) determining that a repositioning of the patient is needed or recommended; and optionally notifying a user thereof visually on an electronic display and/or audibly through a speaker;
(j) determining one or more imaging sequence parameters to be used in the MR imaging of the patient; and optionally implementing the one or more imaging sequence parameters;
(k) determining that an interventional instrument to be used with the positional aid device has been engaged with a thru-passage of the positional aid device other than a thru-passage of the positional aid device that has been determined as acceptable for introduction of the interventional instrument therethrough to access a tissue region of interest in the patient; and optionally notifying a user thereof visually on an electronic display and/or audibly through a speaker;
(l) determining in real time a distance of an interventional instrument from a tissue region of interest as the interventional instrument extends through a thru-passage of the positional aid device and moves relative to the tissue region of interest; and optionally notifying a user thereof in real time visually on an electronic display and/or audibly through a speaker;
(m) determining in real time that interventional instrument moving through tissue of the patient has deviated significantly from a target pathway from a thru-passage of the positional aid device to a tissue region of interest in the patient; and optionally notifying a user thereof in real time visually on an electronic display and/or audibly through a speaker; and/or
(n) determining in real time that interventional instrument extending through a thru-passage of the positional aid device and moving through tissue of the patient has reached a tissue region of interest in the patient; and optionally notifying a user thereof in real time visually on an electronic display and/or audibly through a speaker.
   136. The system of embodiment 135, wherein the MRI system is configured to perform at least operation (f), and wherein said determining a thru-passage is based at least in part on determining a limited range of angles of an interventional instrument available when using the interventional instrument positioned through a thru-passage of the positional aid device.
   137. The system of embodiment 136, wherein the MRI system is configured to perform at least operation (b), and wherein said determining a limited range of angles occurs in operation (b).
   138. The system of embodiment 135, wherein the MRI system is configured to perform at least operation (b), and wherein said one or attributes includes a limited range of angles of an interventional instrument available when using the interventional instrument positioned through a thru-passage of the positional aid device.
   139. The system of any one of embodiments 135 to 138, wherein the positional aid device is a device according to any one of embodiments 1 to 29, 47 to 55, or 160 to 178.
   140. The system of any one of embodiments 135 to 139, also comprising the positional aid device.
   141. A method according to any one of embodiments 130 to 134 wherein said conducting comprises conducting at least step (a), or a system according to any one of embodiments 135 to 139 wherein the MRI system is configured to conduct operation (a).
   142. A method according to any one of embodiments 130 to 134 or 141 wherein said conducting comprises conducting at least step (a), or a system according to any one of embodiments 135 to 139 or 141 wherein the MRI system is configured to conduct operation (a).
   143. A method according to any one of embodiments 130 to 134 or 141 to 142 wherein said conducting comprises conducting at least step (b), or a system according to any one of embodiments 135 to 139 or 141 to 142 wherein the MRI system is configured to conduct operation (b).
   144. A method according to any one of embodiments 130 to 134 or 141 to 143 wherein said conducting comprises conducting at least step (c), or a system according to any one of embodiments 135 to 139 or 141 to 143 wherein the MRI system is configured to conduct operation (c).
   145. A method according to any one of embodiments 130 to 134 or 141 to 144 wherein said conducting comprises conducting at least step (d), or a system according to any one of embodiments 135 to 139 or 141 to 144 wherein the MRI system is configured to conduct operation (d).
   146. A method according to any one of embodiments 130 to 134 or 141 to 145 wherein said conducting comprises conducting at least step (e), or a system according to any one of embodiments 135 to 139 or 141 to 145 wherein the MRI system is configured to conduct operation (e).
   147. A method according to any one of embodiments 130 to 134 or 141 to 146 wherein said conducting comprises conducting at least step (f), or a system according to any one of embodiments 135 to 139 or 141 to 146 wherein the MRI system is configured to conduct operation (f).
   148. A method according to any one of embodiments 130 to 134 or 141 to 147 wherein said conducting comprises conducting at least step (g), or a system according to any one of embodiments 135 to 139 or 141 to 147 wherein the MRI system is configured to conduct operation (g).
   149. A method according to any one of embodiments 130 to 134 or 141 to 148 wherein said conducting comprises conducting at least step (h), or a system according to any one of embodiments 135 to 139 or 141 to 148 wherein the MRI system is configured to conduct operation (h).
   150. A method according to any one of embodiments 130 to 134 or 141 to 149 wherein said conducting comprises conducting at least step (i), or a system according to any one of embodiments 135 to 139 or 141 to 149 wherein the MRI system is configured to conduct operation (i).
   151. A method according to any one of embodiments 130 to 134 or 141 to 150 wherein said conducting comprises conducting at least step (j), or a system according to any one of embodiments 135 to 139 or 141 to 150 wherein the MRI system is configured to conduct operation (j).
   152. A method according to any one of embodiments 130 to 134 or 141 to 151 wherein said conducting comprises conducting at least step (k), or a system according to any one of embodiments 135 to 139 or 141 to 151 wherein the MRI system is configured to conduct operation (k).
   153. A method according to any one of embodiments 130 to 134 or 141 to 152 wherein said conducting comprises conducting at least step (l), or a system according to any one of embodiments 135 to 139 or 141 to 152 wherein the MRI system is configured to conduct operation (l).
   154. A method according to any one of embodiments 130 to 134 or 141 to 153 wherein said conducting comprises conducting at least step (m), or a system according to any one of embodiments 135 to 139 or 141 to 153 wherein the MRI system is configured to conduct operation (m).
   155. A method according to any one of embodiments 130 to 134 or 141 to 154 wherein said conducting comprises conducting at least step (n), or a system according to any one of embodiments 135 to 139 or 141 to 154 wherein the MRI system is configured to conduct operation (n).
   156. A method for preparing a patient for MR imaging, comprising:

positioning an MR imaging coil device according to any one of embodiments 116 to 129 adjacent to the patient.

157. The method of embodiment 156, also comprising obtaining MR imaging data of at least a region of the patient using the MR imaging coil device.
158. The method of embodiment 157, also comprising obtaining MR imaging data of the positional aid device.
159. The method of embodiment 158, also comprising displaying an MR image including the positional aid device and the region of the patient.
160. The magnetic resonance imaging (MRI) positional aid device of embodiment 1, wherein each of the thru-passages is demarked by an identifying portion positioned around the thru-passage, the identifying portion defining geometric shape having a surface visible under MRI.
161. The device of embodiment 160, wherein the plurality of thru-passages are in a defined array.
162. The device of any one of embodiments 160 or 161 wherein each thru-passage of the plurality of thru-passages forms a first concavity of decreasing dimension as it extends from the first side toward the second side, preferably wherein the first concavity is bow-shaped or cone-shaped.
163. The device of embodiment 162 wherein each thru-passage of the plurality of thru-passages forms a second concavity of increasing dimension as it extends from the first side toward the second side.
164. The device of any one of embodiments 160 to 163, wherein the flexible layer structure comprises an MRI visible polymeric material.
165. The device of embodiment 164, wherein the MRI visible polymeric material comprises silicone, a polyurethane, a polystyrene, or a natural or synthetic rubber.
166. The device of any one of embodiments 160 to 165, wherein at least two of the identifying portions define respective geometric shapes that differ from one another.
167. The device of any one of embodiments 160 to 165, wherein the array is a grid array having a first axis and a second axis perpendicular to the first axis.
168. The device of embodiment 167, wherein the grid comprises four quadrants, and wherein the identifying portions in each quadrant define a geometric shape unique to that quadrant.
169. The device of any one of embodiments 160 to 168, wherein each of the identifying portions define a concavity on the first surface defining the geometric shape.
170. The device of any one of embodiments 160 to 169, wherein each of said plurality of thru-passages includes a rigid member positioned within the thru-passage.
171. The device of embodiment 170, wherein the rigid members comprise a material having a higher durometer than that of the flexible layer structure.
172. The device of any one of embodiments 170 or 171, wherein the rigid member comprises a lumen wall that defines the thru-passage, preferably wherein the lumen surface defines a first concave portion and a second concave portion.
173. The device of any one of embodiments 170 to 172, wherein the rigid member extends from the surface of the geometric shape to the second side of the device.
174. The device of any one of embodiments 160 to 169, wherein the flexible layer structure comprises a base layer and a top layer, wherein the top layer comprises a first material and wherein the base layer comprises a second material, and wherein the second material is more flexible than the first material.
175. The device of embodiment 174, wherein the thru-passages are defined by the top layer, and wherein the material of the top layer defining the thru-passages extends through corresponding openings in the base layer.
176. The device of any one of embodiments 160 to 168, each of the identifying portions comprises a raised portion having a top surface defining the geometric shape, and wherein each through passage extends from the top surface of a raised identifying portion to the second side of the device.
177. The device of embodiment 176, wherein the raised identifying portions extend from a base layer, each raised identifying portion having a height from the base later to the top surface, and wherein the height of the raised identifying portions is 1.5 mm to 4 mm.
178. The device of any embodiment 177, wherein the base layer has a thickness of 0.75 mm to 2 mm.
179. A method for using a positional aid device, comprising:

providing a positional aid device according to any one of embodiments 160 to 178; and

performing magnetic resonance (MR) imaging of a target imaging region including the device, the MR imaging generating and displaying at least one MR image in which said thru-passages are demarked in the MR image by the MRI visible material

180. The method of embodiment 179, further comprising:

engaging an interventional device with a contact surface of the flexible layer structure that is adjacent to one of the thru-passages.

181. The method of embodiment 180, wherein the engaging includes contacting a contact surface of the interventional device with a contact surface of the flexible layer structure; optionally wherein the contact surface of the interventional device is contoured to conform to the contact surface of the flexible layer structure.
182. The method of embodiment 181, wherein the contact surface of the interventional device and the contact surface of the flexible layer cooperate to provide an advancement stop to the interventional device such that the interventional device penetrates only partially into the thickness of the positional aid device during said engaging.
183. The method of any one of embodiments 179 to 182, wherein the interventional device comprises an MRI visible material, and wherein the target imaging region includes the interventional device and the at least one MR image includes the interventional device engaged with the contact surface of the flexible layer structure.
184. The method of any one of embodiments 179 to 183, wherein the interventional device comprises a tissue penetrating device, preferably wherein the tissue penetrating device comprises a needle.
185. The method of any one of embodiments 179 to 184, wherein during said performing MR imaging the positional aid device is received adjacent to tissue of a patient, the method further including moving a tissue penetrating device along the insertion path so as to insert the tissue penetrating device into the patient.
186. The method of embodiment 185, which is a biopsy method, the method also including obtaining a tissue sample from an internal region in the patient with the tissue penetrating device.
187. The method of any one of embodiments 179 to 186, wherein said MR imaging is real-time MR imaging and said generating and displaying at least one MR image includes generating and displaying real-time MR images.

1-39. (canceled)

40. A method for using a positional aid device, comprising:
providing a positional aid device according to claim 61; and
performing magnetic resonance (MR) imaging of a target imaging region including the device, the MR imaging generating and displaying at least one MR image in which said thru-passages are demarked in the MR image by the MRI visible material.

41. The method of claim 40, further comprising:
engaging an interventional device with a contact surface of the layer structure that is adjacent to one of the plurality of thru-passages.

42-56. (canceled)

57. The method of claim 40 wherein said MR imaging is real-time MR imaging and said generating and displaying at least one MR image includes generating and displaying real-time MR images.

58-60. (canceled)

61. A positional aid device for use with a medical imaging system, comprising:
a layer structure having a first side and a second side opposite the first side; and
the layer structure defining a plurality of thru-passages;
wherein the thru-passages extend completely through a thickness of the layer structure;
wherein the thru-passages are demarked by a visible material of the layer structure visible in images generated by the medical imaging system; and
wherein the thru-passages of the plurality of thru-passages each form a first concavity at the first side of decreasing dimension as it extends from the first side toward the second side.

62. (canceled)

63. The device of claim 60, wherein the thru-passages of the plurality of thru-passages are in a defined array, wherein the visible indicia include directly visible indicia and/or indicia visible in images generated by the medical imaging system; optionally wherein a first set of the visible indicia is correlated to a first axis of the array and a second set of visible indicia is correlated to a second axis of the array.

64-65. (canceled)

66. The device of claim 61, wherein the medical imaging system is a magnetic resonance imaging system or an X-ray imaging system.

67. The device of claim 61, wherein the layer structure has a thickness of at least about 0.5 cm, or in the range of about 0.5 cm to about 2 cm; and/or wherein the thru-passages of the plurality of thru-passages each form a second concavity of decreasing dimension as it extends from the second side toward the first side.

68-73. (canceled)

74. The device of claim 61, wherein the visible material is contained within one or more internal cavities defined in the layer structure, optionally wherein the one or more internal cavities form a fluidly continuous internal passageway system containing amounts of the visible material demarking at least first and second thru-passages of the plurality of thru-passages, preferably wherein the fluidly continuous internal passageway system contains amounts of the visible material demarking all of the plurality of thru-passages.

75-84. (canceled)

85. The device of claim 61, wherein the first concavity is bowl-shaped or cone-shaped, and/or wherein the second concavity is bowl-shaped or cone-shaped.

86-98. (canceled)

99. A procedural system, comprising:
(a) a positional aid device according to claim 61; and
(b) a guide device for guiding an interventional instrument, the guide device comprising a visible material visible in images generated by the medical imaging system.

100. (canceled)

101. The system of claim 99, wherein the visible material of the guide device demarks an elongate longitudinal path along a length of the guide device, and/or wherein the interventional instrument is a tissue penetrating device, and wherein the guide device is configured to guide the tissue penetrating device along an insertion path.

102-110. (canceled)

111. The system of claim 99, wherein the guide device has a distal tip contoured to conform with respective surface portions of the first side surrounding the thru-passages of the plurality of thru-passages; optionally wherein the distal tip is a convexly rounded distal tip.

112. A method of using a procedural system, comprising:
providing a system according to claim 99; and
engaging the guide device with a surface portion on the first side surrounding a selected one of the plurality of thru-passages.

113. The method of claim 112, also comprising generating an image with the medical imaging system while the guide device is engaged with the surface portion on the first side.

114. (canceled)

115. A medical kit, comprising:
a system according to claim 99; and
a kit package containing the system.

116. An MR imaging coil device configured for use with a positional aid device, comprising:
a flexible base layer having a thru-opening;
a loop antenna positioned in the base layer, optionally around the thru-opening;
at least one mounting frame attached to the base layer and defining a frame opening aligned with the thru-opening of the base layer and configured to receive a positional aid device;
wherein the MRI imaging coil device, and preferably the at least one mounting frame thereof, includes at least one fixation actuator movable between a first position configured to fix the positional aid device to the mounting frame while received in the frame opening and a second position configured to unfix the positional aid device from the mounting frame.

117. The MR imaging coil device of claim 116, wherein the fixation actuator includes at least one projecting member configured for contacting the positional aid device when the fixation actuator is moved from the first position to the second position; optionally wherein the at least one projecting member is configured for receipt within a fixation opening of the positional aid device when the fixation actuator is moved from the first position to the second position.

118-129. (canceled)

130. A method for MR imaging of a patient, comprising:
acquiring MR imaging data of a region including a positional aid device adjacent a patient with an MR imaging system; and
based at least in part on the MR imaging data, conducting at least one of, at least two of, at least three of, or all of, the following steps, using a computer processor:
(a) determining the presence of the positional aid device;
(b) determining one or more attributes of the positional aid device;
(c) determining an orientation of the positional aid device;
(d) determining a distance from a feature of the positional aid device to a tissue region of interest within the patient; and optionally displaying on an electronic display a numeric value representative of said distance from a feature;
(e) determining a target pathway from a feature of the positional aid device to a tissue region of interest within the patient, optionally wherein the feature is a thru-passage of the positional aid device selected from a plurality of thru-passages of the positional aid device; and optionally displaying on an electronic display a graphic representing the target pathway;
(f) determining a thru-passage of the positional aid device selected from a plurality of thru-passages of the positional aid device, acceptable for introducing an interventional instrument therethrough to access a tissue region of interest in the patient; and optionally displaying on an electronic display one or more alphanumeric characters and/or one or more graphics identifying the selected thru-passage;
(g) determining an instrument parameter for accessing a tissue region of interest within the patient through a thru-passage of the positional aid device;
(h) determining that a repositioning of the positional aid device relative to the patient is needed or recommended; and optionally notifying a user thereof visually on an electronic display and/or audibly through a speaker;
(i) determining that a repositioning of the patient is needed or recommended; and optionally notifying a user thereof visually on an electronic display and/or audibly through a speaker;
(j) determining one or more imaging sequence parameters to be used in the MR imaging of the patient; and optionally implementing the one or more imaging sequence parameters;
(k) determining that an interventional instrument to be used with the positional aid device has been engaged with a thru-passage of the positional aid device other than a thru-passage of the positional aid device that has been determined as acceptable for introduction of the interventional instrument therethrough to access a tissue region of interest in the patient; and optionally notifying a user thereof visually on an electronic display and/or audibly through a speaker;
(l) determining in real time a distance of an interventional instrument from a tissue region of interest as the interventional instrument extends through a thru-passage of the positional aid device and moves relative to the tissue region of interest; and optionally notifying a user thereof in real time visually on an electronic display and/or audibly through a speaker;
(m) determining in real time that interventional instrument moving through tissue of the patient has deviated significantly from a target pathway from a thru-passage of the positional aid device to a tissue region of interest in the patient; and optionally notifying a user thereof in real time visually on an electronic display and/or audibly through a speaker; or
(n) determining in real time that interventional instrument extending through a thru-passage of the positional aid device and moving through tissue of the patient has reached a tissue region of interest in the patient; and optionally notifying a user thereof in real time visually on an electronic display and/or audibly through a speaker.

131. The method of claim 130, wherein said conducting comprises conducting at least step (f), and wherein said determining a thru-passage is based at least in part on determining a limited range of angles of an interventional instrument available when using the interventional instrument positioned through a thru-passage of the positional aid device.

132. The method of claim 131, also comprising conducting at least step (b), and wherein said determining a limited range of angles occurs in step (b).

133-134. (canceled)

135. A system for imaging of a patient, comprising:
a magnetic resonance imaging (MRI) system configured to acquire MRI data of a region including a positional aid device adjacent a patient; and
wherein the MRI system is configured to perform, based at least in part on the MRI data, at least one of, at least two of, at least three of, or all of, the following operations, using a computer processor:
(a) determining the presence of the positional aid device;
(b) determining one or more attributes of the positional aid device;
(c) determining an orientation of the positional aid device;
(d) determining a distance from a feature of the positional aid device to a tissue region of interest within the patient; and optionally displaying on an electronic display a numeric value representative of said distance from a feature
(e) determining a target pathway from a feature of the positional aid device to a tissue region of interest within the patient, optionally wherein the feature is a thru-passage of the positional aid device selected from a plurality of thru-passages of the positional aid device; and optionally displaying on an electronic display a graphic representing the target pathway;
(f) determining a thru-passage of the positional aid device selected from a plurality of thru-passages of the positional aid device, acceptable for the introduction of an interventional instrument therethrough to access a tissue region of interest in the patient; and optionally displaying on an electronic display one or more alphanumeric characters and/or one or more graphics identifying the selected thru-passage;
(g) determining an instrument parameter for accessing a tissue region of interest within the patient through a thru-passage of the positional aid device;
(h) determining that a repositioning of the positional aid device relative to the patient is needed or recommended; and optionally notifying a user thereof visually on an electronic display and/or audibly through a speaker;
(i) determining that a repositioning of the patient is needed or recommended; and optionally notifying a user thereof visually on an electronic display and/or audibly through a speaker;
(j) determining one or more imaging sequence parameters to be used in the MR imaging of the patient; and optionally implementing the one or more imaging sequence parameters;
(k) determining that an interventional instrument to be used with the positional aid device has been engaged with a thru-passage of the positional aid device other than a thru-passage of the positional aid device that has been determined as acceptable for introduction of the interventional instrument therethrough to access a tissue region of interest in the patient; and optionally notifying a user thereof visually on an electronic display and/or audibly through a speaker;
(l) determining in real time a distance of an interventional instrument from a tissue region of interest as the interventional instrument extends through a thru-passage of the positional aid device and moves relative to the tissue region of interest; and optionally notifying a user thereof in real time visually on an electronic display and/or audibly through a speaker;
(m) determining in real time that interventional instrument moving through tissue of the patient has deviated significantly from a target pathway from a thru-passage of the positional aid device to a tissue region of interest in the patient; and optionally notifying a user thereof in real time visually on an electronic display and/or audibly through a speaker; and/or
(n) determining in real time that interventional instrument extending through a thru-passage of the positional aid device and moving through tissue of the patient has reached a tissue region of interest in the patient; and optionally notifying a user thereof in real time visually on an electronic display and/or audibly through a speaker.

136. The system of claim 135, wherein the MRI system is configured to perform at least operation (f), and wherein said determining a thru-passage is based at least in part on determining a limited range of angles of an interventional instrument available when using the interventional instrument positioned through a thru-passage of the positional aid device.

137. The system of claim 136, wherein the MRI system is configured to perform at least operation (b), and wherein said determining a limited range of angles occurs in operation (b).

138-155. (canceled)

156. A method for preparing a patient for MR imaging, comprising:
positioning an MR imaging coil device according to claim 116 adjacent to the patient, and optionally comprising obtaining MR imaging data of at least a region of the patient using the MR imaging coil device.

157-159. (canceled)

160. The positional aid device of claim 61, wherein each of the thru-passages is demarked by an identifying portion positioned around the thru-passage, the identifying portion defining geometric shape having a surface visible under MRI.

161-163. (canceled)

164. The device of claim 61, wherein the layer structure comprises a flexible layer structure comprising an MRI visible polymeric material, optionally wherein the MRI visible polymeric material comprises silicone, a polyurethane, a polystyrene, or a natural or synthetic rubber.

165. (canceled)

166. The device of claim 160, wherein at least two of the identifying portions define respective geometric shapes that differ from one another.

167. The device of claim 160, wherein the array is a grid array having a first axis and a second axis perpendicular to the first axis, and wherein the grid comprises four quadrants, and wherein the identifying portions in each quadrant define a geometric shape unique to that quadrant.

168-169. (canceled)

170. The device of claim 160, wherein each of said plurality of thru-passages includes a rigid member positioned within the thru-passage.

171. (canceled)

172. The device of claim 170, wherein the rigid member comprises a lumen wall that defines the thru-passage, preferably wherein the lumen surface defines a first concave portion and a second concave portion.

173. (canceled)

174. The device of claim 160, wherein the layer structure comprises a base layer and a top layer, wherein the top layer comprises a first material and wherein the base layer comprises a second material, and wherein the second material is more flexible than the first material.

175. (canceled)

176. The device of claim 160, wherein each of the identifying portions comprises a raised portion having a top surface defining the geometric shape, and wherein each through passage extends from the top surface of a raised identifying portion to the second side of the device.

177-187. (canceled)
